(19) United States
(12) Patent Application Publication
(10) Pub. No.: US 2016/0279251 A1
(43) Pub. Date: Sep. 29, 2016

(54) RANDOM COPOLYMER THERAPEUTIC AGENT CARRIERS AND ASSEMBLIES THEREOF

(71) Applicant: UNIVERSITY OF WASHINGTON THROUGH ITS CENTER FOR COMMERCIALIZATION, Seattle, WA (US)

(72) Inventors: Patrick S. Stayton, Seattle, WA (US); Anthony Convertine, Seattle, WA (US); Daniel M. Ratner, Seattle, WA (US); Selvi Srinivasan, Seattle, WA (US); Debobrato Das, Seattle, WA (US); Fang-Yi Su, Seattle, WA (US); Jasmin Chen, Seattle, WA (US); David Yee-Shawn Chiu, Seattle, WA (US); Daniel Douglas Lane, Seattle, WA (US)

(73) Assignee: University of Washington through its Center for Commercialization, Seattle, WA (US)

(21) Appl. No.: 15/035,721

(22) PCT Filed: Nov. 12, 2014
(86) PCT No.: PCT/US2014/065292

§ 371 Date: May 10, 2016

Related U.S. Application Data
(60) Provisional application No. 61/903,313, filed on Nov. 12, 2013.

(51) Int. Cl. A61K 47/48; A61K 31/407; A61K 31/4745; A61K 31/7036; A61K 31/337; A61K 31/496

(52) U.S. Cl. CPC A61K 47/48176; A61K 47/483; A61K 31/337; A61K 47/48215; A61K 47/488; A61K 31/496; A61K 31/4745; A61K 31/7036; A61K 31/407; A61K 47/4883

(57) ABSTRACT

Provided herein are particles assemblies including a shell surrounding a core. The shell includes a particle-stabilizing random copolymer. The core includes a core random copolymer. The particle assemblies have a biomimetic design in which the polymeric components containing discrete chemical and biological functionalities are designed to spontaneously self-assemble into particles. Also provided herein are random copolymers having conjugated therapeutic agents that can be cleaved from the copolymers by an enzyme or water.

### CROSS-REFERENCE TO RELATED APPLICATION

This application claims the benefit of U.S. Patent Application No. 61/903,313, filed Nov. 12, 2013, the disclosure of which is hereby incorporated by reference in its entirety.

### BACKGROUND

A major hurdle in the realm of pharmaceutical and medicinal chemistry is the ability to deliver biologically effective drugs to a targeted area of the body, with minimal toxicity. For example, chemotherapy is a frontline approach to managing cancer but is associated with a range of serious dose-limiting toxicities including cardiomyopathy, febrile neutropenia, anemia, and thrombocytopenia. The use of nanoparticle-based therapies to deliver cytotoxic agents has the potential to significantly improve the activity and side-effect profiles. Chemotherapeutic nanoparticle formulations such as Doxil (liposomal encapsulated doxorubicin) exhibit enhanced circulation half-lives (up to 100 times greater than the unencapsulated drug) yet cause substantially lower deleterious side effects. In the case of Doxil, the risk of cardiotoxicity is 7-fold lower than the free drug despite the large difference in circulation half-lives.

The application of controlled radical polymerization (CRP) technology to prepare drug delivery systems has allowed the discovery of new material systems. For example, block copolymers of N-(2-hydroxypropyl) methacrylamide (HPMA) with a bioconjugatable monomer 2-(2-pyridyldisulfide) ethylmethacrylate (PDSMA) via reversible addition-fragmentation chain transfer (RAFT) polymerization have been described. The resultant diblock copolymer was conjugated to doxorubicin and crosslinked via hydrazone linkages to form micellar assemblies that released free drug upon a decrease in pH. The sequestration of hydrophilic platinum-based therapeutics has been achieved via a combination of RAFT, thiol-ene, and thiol-yne chemistry and yielded materials with pendent Pt drugs. Specifically, platinum delivery systems based on block copolymer micelles, which consist of a hydrophilic biocompatible polyethylene glycol methacrylate (PEGMA) corona and a hydrophobic styrene core containing reactive isocyanate groups for conjugation of cisplatin prodrug, have been described.

Targeted therapies have the potential to further enhance the effectiveness of therapies using nano and microparticles. Active targeting of tumor surface receptors via antibodies, peptides, aptamers, and vitamins can significantly enhance the specificity of these therapies compared to passive targeting alone. For example, chemotherapeutic delivery systems may be coupled to targeting agents through the use of activated ester-containing compounds resulting in conjugates linked via stable amide bonds. Activated esters include, for example, succinimidyl and pentafluorophenyl esters as well as mercaptothiazoline.

Efficient targeted delivery of therapeutic agents can also be important in the prevention and treatment of infections. For example, the dissemination of infectious aerosols is considered the most dangerous method of delivering biological weapons. Francisella tularensis and Burkholderia pseudomallei, the agents of tularemia and melioidosis, respectively, are among the Center for Disease Control's Tier 1 select agents because they present the greatest risk of mass casualties or devastating effects to the economy, critical infrastructure, and public confidence. Both F. tularensis and B. pseudomallei are environmentally stable, can be transmitted by aerosol, and can cause a rapid onset of severe illness. Both infections are most lethal when acquired by inhalation. The intracellular compartmentalization of these pathogenic organisms within alveolar macrophages is a significant barrier to bacterial clearance and contributes to their associated morbidity and mortality. Because rapid diagnostic tests for tularemia and melioidosis are not readily available, rapid prophylactic/treatment methods are all the more important, since even effective antibiotics do not prevent disabling illness when treatment is started after the onset of symptoms.

To reach the relevant alveolar macrophage sub-compartments necessary to open prophylactic and presymptomatic therapy strategies, therapeutic agents, such as antibiotics, must be ferried across a complex and rugged biological terrain over long distances, while evading biological mechanisms that are designed to degrade, inactivate or clear the molecules. Crossing these barriers at the system-wide, tissue, cellular and sub-cellular levels is the key to the ultimate efficacy, and one that in broad terms continues to stymie the field.

Accordingly, there is need for drug delivery compositions that can provide efficient drug delivery with minimal toxicity, for example, in areas such as chemotherapy and antibiotic treatment (e.g., pre- and post-exposure protection to personnel in the settings of a known/suspected airborne exposure to biothreat agents, such as F. tularensis and B. pseudomallei). The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, this disclosure features a particle assembly including a shell and a core. The shell surrounds the core. The shell can include a particle-stabilizing random copolymer including (i) hydrophobic uncharged constitutional units, cationic constitutional units, anionic constitutional units, or any combination thereof, (ii) hydrophilic uncharged constitutional units, and (iii) a covalently-bound targeting agent. The core includes a core random copolymer including (i) hydrophobic uncharged constitutional units; (ii) cationic constitutional units, anionic constitutional units, or both cationic constitutional units and anionic constitutional units; and (iii) a covalently-bound therapeutic agent.

In another aspect, this disclosure features a random copolymer including at least one constitutional unit selected from cationic constitutional units, anionic constitutional units, any combination thereof, and hydrophobic uncharged constitutional units, and constitutional units comprising a covalently-bound antibiotic agent or kinase inhibitor. The antibiotic agent or kinase inhibitor is cleavable from the copolymer by an enzyme or water.

In yet another aspect, this disclosure features a random copolymer including (i) hydrophobic uncharged constitutional units, cationic constitutional units, or anionic constitutional units, or any combination thereof, (ii) hydrophilic constitutional units, and (iii) constitutional units comprising a covalently-bound antibiotic agent or kinase inhibitor. The antibiotic agent or kinase inhibitor is cleavable from the copolymer by an enzyme or water.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a graphical representation of an embodiment of a particle assembly of the present disclosure;

FIG. 2 is a graphical representation of an embodiment of a particle assembly of the present disclosure entering an alveolar macrophage;

FIGS. 3A and 3B are graphical representations of a polymer-augmented liposome before and after disassembly;

FIG. 4 is an illustration of an embodiment of a therapeutic agent-conjugated polymer and hydrolysis rates of embodiments of linkages;

FIG. 5A is an illustration of embodiments of therapeutic agent-conjugated monomers;

FIG. 5B is an illustration of embodiments of therapeutic agent-conjugated monomers or constitutional units;

FIG. 5C is an illustration of embodiments of therapeutic agent conjugation methods;

FIG. 6 is a schematic representation of an embodiment of a copolymer particle assembly including tumor specific transferrin targeting groups, a hydrophilic polyethylene glycol (PEG) shell, and a hydrophobic drug-sequestering core;

FIGS. 7A-7C are graphs showing the molecular weight, composition, and critical micelle concentration values for poly(LMA_{co}O950_{co}TMA) prepared by RAFT. FIG. 7A is a representative SEC chromatogram (refractive index channel) showing the narrow and symmetric molecular weight distribution for a copolymer of PEGMA 950, LMA, and TMA (25:50:25) feed ratio. FIG. 7B is a ¹H NMR spectrum of the copolymer in CDCl₃ with assignment of the resonances associated with the respective comonomer residues. FIG. 7C is a graph showing rhodamine 6G fluorescence as a function of copolymer composition and concentration in phosphate buffered saline (20 mM sodium phosphate, 150 mM NaCl; Ph 7.4) with an excitation and emission of 480 and 550 nm respectively;

FIGS. 8A-8C are live cell deconvoluted fluorescence microscopy images of HeLa cells treated with fluorescently-labeled Transferrin-polymer conjugates. FIG. 8A shows fluorescence from AlexaFluor 488-labeled transferrin-polymer conjugates. FIGS. 8B and 8C shows HeLa cells treated with nuclear stain (Hoechst) and plasma membrane (Cellmask deep red) administered 30 minutes and 2 minutes prior to analysis, respectively;

FIG. 9 is a graph showing cytotoxicity of a random copolymer-encapsulated docetaxel evaluated in HeLa (human cervical cancer) cells. Formulations were prepared over a range of docetaxel concentrations between 1 and 100 nM. Dose response curves are for cells at 72 hours post-treatment with polymer-encapsulated drug with and without Transferrin targeting. Cell viability numbers are relative to untreated controls as determined by MTS. Data represent the average of 2 experiments conducted in quadruplicate;

FIG. 10A is a schematic representation of a polymer having a slow-releasing conjugated therapeutic agent;

FIG. 10B is a schematic representation of a polymer having a fast-releasing conjugated therapeutic agent;

FIG. 11A is an illustration of the synthesis of a binary streptomycin-conjugated random copolymer;

FIG. 11B is an illustration of the synthesis of a ciprofloxacin-conjugated random copolymer;

FIG. 11C is an illustration of a particle assembly formed from the streptomycin-conjugated random copolymer and ciprofloxacin-conjugated random copolymer of FIGS. 11A and 11C; and

FIG. 11D is a schematic representation of the assembly and disassembly of the polymer assembly of FIG. 11C.

### DETAILED DESCRIPTION

The present disclosure provides particles assemblies including a shell surrounding a core. The shell includes a particle-stabilizing random copolymer including constitutional units (i), (ii), and (iii), where constitutional units (i) can include hydrophobic uncharged constitutional units, cationic constitutional units, anionic constitutional units, or any combination thereof; constitutional units (ii) can include hydrophilic uncharged constitutional units; and constitutional units (iii) can include a covalently-bound targeting agent. The core includes a core random copolymer including constitutional units (i), (ii), and (iii), where constitutional units (i) can include hydrophobic uncharged constitutional units; constitutional units (ii) can include cationic constitutional units, anionic constitutional units, or both cationic constitutional units and anionic constitutional units, and constitutional units (iii) can include a covalently-bound therapeutic agent. The particle assemblies have a biomimetic design in which the polymeric components containing discrete chemical and biological functionalities are designed to spontaneously self-assemble into particles. This self-assembly process can be driven by hydrophobic interactions between the biocompatible particle-stabilizing random copolymer and the core random copolymer.

The present disclosure also provides random copolymers, including (i) cationic constitutional units, anionic constitutional units, or any combination thereof, and/or (ii) hydrophobic uncharged constitutional units, and (iii) constitutional units including a covalently-bound antibiotic agent or kinase inhibitor. The antibiotic agent or kinase inhibitor is cleavable from the copolymer by an enzyme or water.

The present disclosure also provides random copolymers, including (i) hydrophobic uncharged constitutional units, cationic constitutional units, or anionic constitutional units, or any combination thereof, (ii) hydrophilic constitutional units, and (iii) constitutional units including a covalently-bound antibiotic agent or kinase inhibitor. The antibiotic agent or kinase inhibitor is cleavable from the copolymer by an enzyme or water.

Compared to block copolymers, the random copolymers of the present disclosure are easy to prepare. The random copolymers can be easily tailored to suit various hydrophobic and/or hydrophilic environments, and can be conjugated to a variety of therapeutic agents and/or targeting agents. The constitutional units and the amount of constitutional units of the random copolymers can be easily adjusted to provide a desired release profile of a conjugated or an encapsulated therapeutic agent in a physiological environment.

### DEFINITIONS

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkenyl” refers to an alkenyl group having one or more halogen substituents.

As used herein, “haloalkynyl” refers to an alkynyl group having one or more halogen substituents.

As used herein, “haloalkoxy” refers to an —O-(haloalkyl) group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “arylene” refers to a linking aryl group.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like.

As used herein, “cycloalkylene” refers to a linking cycloalkyl group.

As used herein, “heteroalkyl” refers to an alkyl group having at least one heteroatom such as sulfur, oxygen, or nitrogen.

As used herein, “heteroalkylene” refers to a linking heteroalkyl group.

As used herein, a “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, the term “fatty acid” refers to a molecule having a carboxylic acid with a long aliphatic chain, which is either saturated or unsaturated.

As used herein, the term “fatty acid ester” refers to a long aliphatic chain (saturated or unsaturated) having a —C(O)O— moiety at an end of the chain.

As used herein, the term “fatty acid amide” refers to a long aliphatic chain (saturated or unsaturated) having a —C(O)NR— moiety at an end of the chain.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “hydrodynamic diameter” refers to the apparent size of particle assemblies hydrated in a solvent (e.g., water), as measured by dynamic light scattering.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, a “particle assembly” refers to a particle that includes a random copolymer. For example, the particle assembly can be in the form of a core-shell particle, where at least one of the core or the shell includes a random copolymer of the present disclosure. The particle assembly can be in the form of a solid particle that includes one or more random copolymers. The particle assembly can be a micelle or a vesicle including one or more random copolymers. The particle assembly can be a random copolymer-augmented liposome.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “neutral net charge” is defined as a polymer having a net charge that is less than 50 percent (e.g., less than 20 percent, less than 10 percent, less than 5 percent, or less than 2 percent) of the anionic or cationic groups content on the polymer chain. In some embodiments, a neutral net charge polymer is uncharged.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, “living polymerization” refers to a method of synthesizing polymers using the well-known concept of addition polymerization, that is, polymerization wherein monomers are added one-by-one to an active site on the growing polymer chain but one wherein the active sites for continuing addition of another monomer are never fully eliminated other than on purpose. That is, the polymer chain is virtually always capable of further extension by the addition of more monomer to the reaction mixture unless the polymer has been capped, which may be reversible so as permit polymerization to continue or quenched, which is usually permanent. While numerous genera of living polymerizations are known, currently the predominant types are anionic, cationic, and radical living polymerizations. Radical polymerization involves a free radical initiator that extracts one of the pi electrons of the double bond of an ethylenic monomer resulting in a reactive unpaired electron on the carbon at the other end of the former double bond from that with which the initiator reacted. The unpaired electron then reacts with the double bond of another monomer creating a stable sigma bond and another free radical and so on. With conventional initiators the sequence is eventually stopped by a termination reaction, generally a combination reaction in which the unpaired electrons of two propagating chains combine to form a stable sigma bond or a disproportionation in which a radical on an active chain strips a hydrogen atom from another active chain or from an impurity in the reaction mixture to produce a stable unreactive molecule and a molecule containing a double bond. In a living polymerization, the ability of the growing chains to enter into a termination reaction is eliminated, effectively limiting the polymerization solely by the amount of monomer present; that is, the polymerization continues until the supply of monomer has been exhausted. At this point the remaining free radical species become substantially less active due to capping of the free radical end group with such entities as, without limitation, nitroxyl radicals, halogen molecules, oxygen species such as peroxide and metals or simply by interaction with solvent and the like. If, however, more monomer is added to the solution, the polymerization reaction can resume except as noted above.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Particle Assembly

Core-Shell Particles

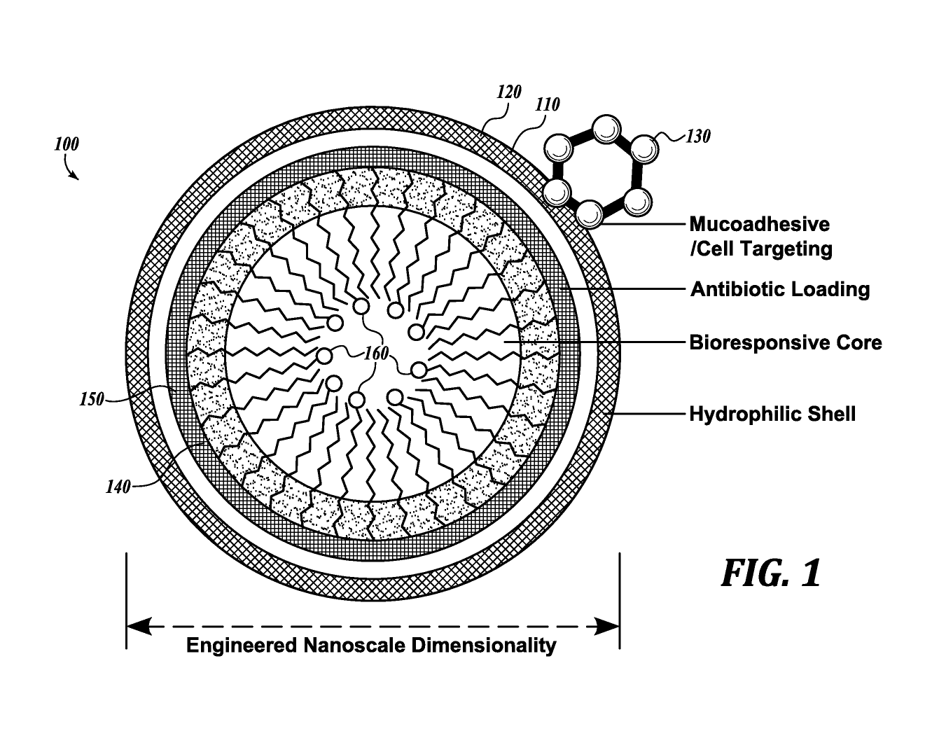

FIG. 1 shows a particle assembly of the present disclosure. Referring to FIG. 1, the particle assembly 100 has an outer shell 110 formed of a particle-stabilizing random copolymer 120. Particle assembly 100 has targeting groups 130 on the surface that are covalently bound to random copolymer 120. Outer shell 110 surrounds inner core 140, which is formed of a core random polymer 150. Core random polymer 150 includes covalently bound therapeutic agents 160.

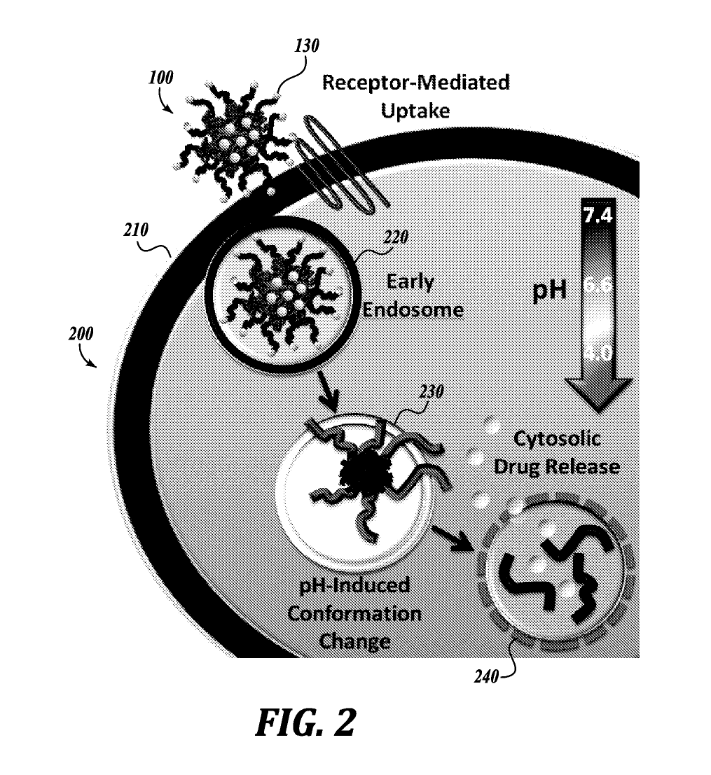

The core random copolymer is relatively hydrophobic compared to the particle-stabilizing random copolymer such that in an aqueous environment, the core random polymer is enveloped by the more hydrophilic particle-stabilizing random copolymer. The particle assembly can mimic the multifunctional delivery systems of pathogens to overcome barriers to delivering therapeutic agents to the cytoplasm of target cells. Referring to FIG. 2, when particle assembly 100 is delivered to a subject, targeting group 130 optimize cell uptake by binding to a cell surface 210 to which it is targeted (e.g., an alveolar macrophage) while minimizing off-target distribution and toxicity, and the particle assembly enters into the cell 200 via receptor-mediated endocytosis. For example, in some embodiments, polyvalent glycan constitutional units in the particle-stabilizing random copolymers can efficiently mediate cell-specific uptake of the therapeutic particle assembly. Once inside the cell, the particle assembly disassembles due to an intracellular vesicular pH drop as an early endosome 220 is transformed into a late endosome 230. As the polymeric components of the particle assembly are exposed, endosomal membrane 240 can be disrupted, and transport of therapeutic agent is thereby enhanced across intracellular vesicular membranes to the cytosol.

Polymer-Augmented Liposomes

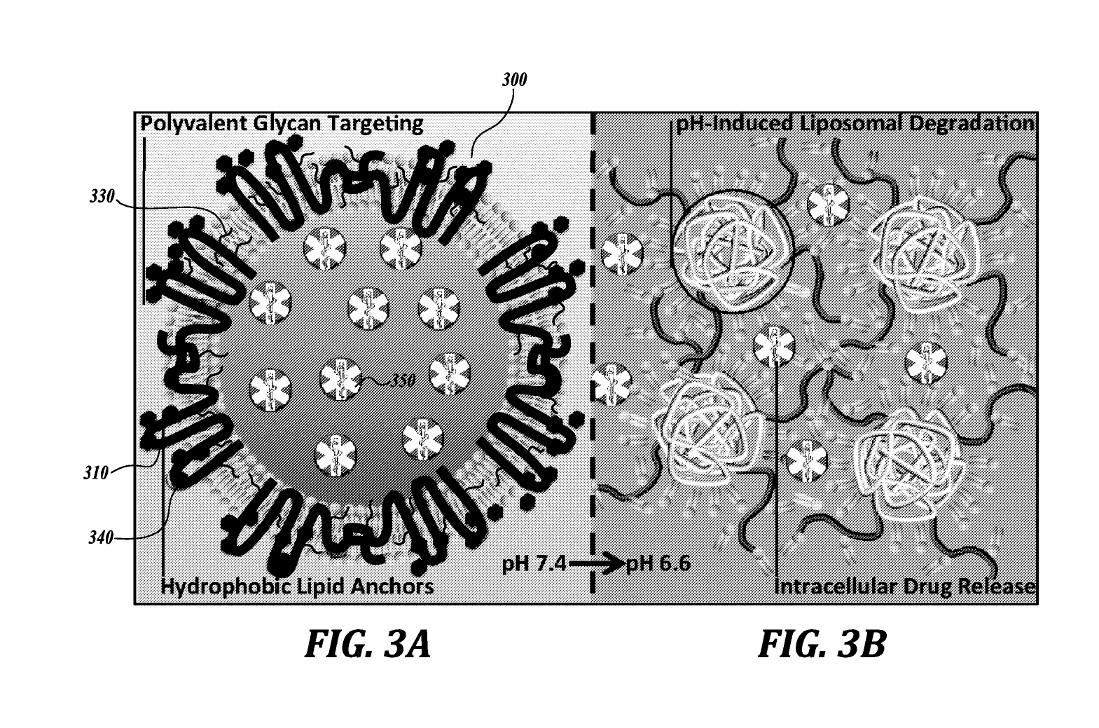

Instead of forming a core-shell particle, in some embodiments, the particle assembly of the present disclosure can be in the form of random copolymers (e.g., the particle-stabilizing copolymer and/or the core random copolymer, as described above) that are incorporated into liposomes to form polymer-augmented liposomes. The polymer-augmented liposome can encapsulate a therapeutic agent and/or can have therapeutic agents that are covalently attached to the random copolymers. For example, referring to FIG. 3A, a polymer-augmented liposome 300 includes pH-responsive polymer constitutional units 310 anchored to a lipid membrane 320 via hydrophobic side chains 330 that insert into lipid membrane 320. Polymer-augmented liposomes 300 can include targeting groups 340 at the surface of the polymer-augmented liposomes to provide cell-specific targeting. Referring to FIG. 3B, when internalized into an endosome, acidification can occur to trigger degradation of the polymer-augmented liposome and cytoplasmic delivery of any encapsulated drugs 350. As an example, a random copolymer including hydrophilic glycan targeting groups can be anchored to a lipid bilayer via lauryl side chains on lauryl methacrylate constitutional units. The random copolymer can also include pH-responsive constitutional units (e.g., cationic and/or anionic constitutional units), which can be present in a hydrophobic lipid bilayer, and serve to impart cytoplasmic delivery and to provide a pH-tunable mechanism for intracellular drug release. Upon internalization into intracellular compartments and subsequent pH-drop, protonation of the cationic constitutional units can destabilize the lipid bilayer and release drug to the cytosol.

The polymer-augmented liposomes can include a random copolymer in an amount of more than 5 percent (e.g., more than 10 percent, more than 20 percent, more than 30 percent, more than 50 percent, or more than 75 percent) and/or less than 95 percent (e.g., less than 75 percent, less than 50 percent, less than 30 percent, less than 20 percent, or less than 10 percent) by weight. In some embodiments, the polymer-augmented liposomes can include 5 percent or more (e.g., 10 percent or more, 20 percent or more, 30 percent or more, 50 percent or more, or 75 percent or more) and/or 95 percent or less (e.g., 75 percent or less, 50 percent or less, 30 percent or less, 20 percent or less, or 10 percent or less) by weight of a phospholipid and/or a cholesterol.

Examples of phospholipids that can form a polymer-augmented liposome include phosphatidylcholines, sphingomyelins, phosphatidic acid (e.g., 1,2-dimyristoyl-sn-glycero-3-phosphate, 1,2-dipalmitoyl-sn-glycero-3-phosphate, 1,2-distearoyl-sn-glycero-3-phosphate), phosphatidylcholine (e.g., 1,2-didecanoyl-sn-glycero-3-phosphocholine, 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1,2-dioleoyl-sn-glycero-3-phosphocholine, 1-palmitoyl-2-oleoyl-sn-glycero-3-phosphocholine 2-dierucoyl-sn-glycero-3-phosphocholine), phosphatidylglycerol, phosphatidylethanolamine (e.g., 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine), phosphatidylserine (e.g., 1,2-dioleoyl-sn-glycero-3-phosphoserine), and PEG-conjugated phospholipid (e.g., mPEG-phospholipid, polyglycerin-phospholipid, functionalized-phospholipid, terminal activated-phospholipid).

### Membrane Disruption

The random copolymers of the present disclosure (e.g., the particle-stabilizing random copolymer, the core random polymer) are disruptive of a cellular membrane, including an extracellular membrane, an intracellular membrane, a vesicle, an organelle, an endosome, a liposome, or a red blood cell. In some embodiments, the random copolymers disrupt the membrane and enter the intracellular environment. In specific examples, the random copolymers are endosomolytic.

In some embodiments, the random copolymers allow for endosomal escape of the therapeutic agent through a pH-induced conformational change which, in some instances, results in membrane destabilization. For example, in some instances, under physiological conditions, a random copolymer has both positive residues and negative residues in similar amounts, resulting in approximate charge neutrality and charge stabilization by formation of ion pairs. Upon uptake of a polymer assembly into endosomal compartments of the cell, the lower pH of the endosomal environment causes anionic residues to become protonated and thereby membrane disruptive. In some instances, a change in the charge of a random copolymer results in a conformational change in the polymer to a hydrophobic membrane-destabilizing form.

Without wishing to be bound by theory, a membrane destabilizing polymer (e.g., a random copolymer of the present disclosure) can directly or indirectly elicit a change (e.g., a permeability change) in a cellular membrane structure (e.g., an endosomal membrane) so as to permit a therapeutic agent, in association with or independent of a polymer, to pass through such membrane structure for example, to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or disruption of a cellular membrane (e.g., as observed for a substantial fraction of a population of cellular membranes).

Generally, membrane destabilizing or membrane disruptive properties of polymers can be assessed by various means. For example, a change in a cellular membrane structure can be observed by assessment in assays that measure (directly or indirectly) release of a therapeutic agent from cellular membranes (e.g., endosomal membranes)—for example, by determining the presence or absence of such agent, or an activity of such agent, in an environment external to such membrane. As another example, red blood cell lysis (hemolysis) can be used as a surrogate assay for a cellular membrane of interest. Such assays may be done at a single pH value or over a range of pH values.

### Physical Characteristics of the Particle Assembly

The particle assembly can be in a variety of forms. For example, the core can be in the form of a solid particle, such that the core is substantially free of cavities and is not-compressible. In some embodiments, the core can be in the form of a micelle that can have a cavity in the center of the micellar structure. In some embodiments, the particle assembly can be a random copolymer-augmented liposome having a cavity in the center of the structure. When the particle assembly has one or more cavities, the cavities can be used to encapsulate a therapeutic agent via, for example, non-covalent interactions. The particle assembly can be sufficiently robust to maintain chemical and conformational stability in physiological solutions.

In some embodiments, at least one of the particle-stabilizing random copolymer and the core random copolymer is biodegradable. For example, the particle-stabilizing random copolymer backbone and/or the core random copolymer polymer backbone can range in size between 10 and 100 kDa but can be broken down into 1-10 kDa segments that can easily be cleared from circulation. In some embodiments, the entire particle assembly is biodegradable.

The particle assembly can have a hydrodynamic diameter of greater than or equal to 8 nm (e.g., greater than or equal to 10 nm, greater than or equal to 25 nm, greater than or equal to 50 nm, greater than or equal to 75 nm, or greater than or equal to 100 nm) and/or less than or equal to 125 nm (e.g., less than or equal to 100 nm, less than or equal to 75 nm, less than or equal to 50 nm, less than or equal to 25 nm, or less than or equal to 10 nm). The small size of the particle assembly allows delivery of the therapeutic agents with better cellular transport, distribution and bioavailability, compared to particles having a hydrodynamic diameter of greater than 125 nm. In some embodiments, the small size of the particle assemblies facilitate delivery of therapeutic agents to regions where larger nanoparticle assemblies (e.g., having a hydrodynamic diameter of greater than 125 nm) cannot penetrate.

To change the size of a particle assembly, the ratio of hydrophilic to hydrophobic constitutional unit in one or more polymer components, the ratio of polymeric stabilizer copolymer and core copolymer, and/or the molecular weight of one or more polymer components, can each independently be varied.

In some embodiments, the particle-stabilizing random copolymer and the core random copolymer have opposite net charges. In some embodiments, the particle-stabilizing random copolymer and/or the core random copolymer have neutral net charge. For example, the particle-stabilizing random copolymer and/or the core random polymer can have near equal amounts of positive and negative charges, or can have constitutional units that are uncharged.

### Particle-Stabilizing Random Copolymer

In some embodiments, the particle-stabilizing copolymer includes hydrophobic uncharged constitutional units. The hydrophobic uncharged constitutional units can each include a C₈-C₂₆ fatty acid side chain. For example, the fatty acid side chain can include unsaturated fatty acid side chains, such as myristoleic acid, palmitoleic acid, sapienic acid, oleic acid, elaidic acid, vaccenic acid, linoleic acid, linoelaidic acid, α-linoelaidic acid, α-linolenic acid, arachidonic acid, eicosapentaenoic acid, erucic acid, or docosahexaenoic acid side chains. As another example, the fatty acid side chain can include saturated fatty acid side chains, such as caprylic acid, capric acid, lauric acid, myristic acid, palmitic acid, stearic acid, arachidic acid, behenic acid, lignoceric acid, or cerotic acid. The fatty acid side chain is covalently bound to the constitutional unit via, for example, an ester linkage or an amide linkage.

In some embodiments, the hydrophobic uncharged constitutional units include a constitutional unit of Formula (I)

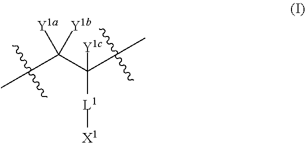

wherein

Y^{1a}, Y^{1b}, and Y^{1c} are each independently selected from H, CN, C₁₋₁₀ alkyl, C₂₋₁₀ alkynyl, C₁₋₁₀ heteroalkyl, cycloalkyl, heterocycloalkyl, aryl, heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L¹ is absent, C₁₋₁₀ alkylene, C(O)OC₁₋₁₀ alkylene, C(O)O(CR¹R²)_{q}, C(O)NR³(CR¹R²)_{q}; (CR¹R²)ₚC(O)O(CR¹R²)_{q}, or (CR¹R²)ₚC(O)NR³(CR¹R²)_{q};

X¹ is H, a fatty acid ester, or a fatty acid amide;

R¹ and R² are each independently H, halo, C₁₋₁₀ alkyl, or C1-10 haloalkyl;

R³ is H, C₁₋₆ alkyl, C₂₋₆ alkenyl, or C₂₋₆ alkynyl;

p is 0, 1, 2, 3, or 4; and

q is 0, 1, 2, 3, or 4.

In some embodiments, Y^{1a}, Y^{1b}, and Y^{1c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{1a}, Y^{1b}, and Y^{1c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{1a}, Y^{1b}, and Y^{1c} are each independently selected from H, and C₁₋₁₀ alkyl.

In some embodiments, Y^{1a}, Y^{1b}, and Y^{1c} are each independently selected from H, methyl, ethyl, and propyl.

In some embodiments, Y^{1a}, Y^{1b}, and Y^{1c} are each independently selected from H and methyl.

In some embodiments, L¹ is absent, C₁₋₁₀ alkylene, C(O)OC₁₋₁₀ alkylene, or C(O)O(CR¹R²)_{q}.

In some embodiments, L¹ is absent, C₁₋₁₀ alkylene, or C(O)OC₁₋₁₀ alkylene.

In some embodiments, X¹ is a fatty acid ester.

In some embodiments, X¹ is H.

In some embodiments, the fatty acid ester or the fatty acid amide includes a C₈₋₂₆ fatty acid chain (e.g., a C₈₋₂₄ fatty acid chain, a C₁₀₋₁₆ fatty acid chain, a C₁₂ fatty acid chain).

In some embodiments, R¹ and R² are each independently H or C₁₋₁₀ alkyl.

In some embodiments, R¹ and R² are each independently H or C₁₋₄ alkyl.

In some embodiments, R³ is H or C₁₋₆ alkyl.

In some embodiments, p is 0.

In some embodiments, p is 1, 2, 3, or 4.

In some embodiments, q is 0.

In some embodiments, q is 1, 2, 3, or 4.

In some embodiments, the particle-stabilizing copolymer includes hydrophilic uncharged constitutional units formed from ethylene glycol-based monomers, ethylene glycol methacrylate, ethylene glycol acrylate, dimethylacrylamide, hydroxyethylacrylamide, and/or 2-hydroxypropyl methacrylamide. In some embodiments, instead of alkylene glycol based monomers, the hydrophilic uncharged constitutional units of the particle-stabilizing random copolymer can be formed of saccharide-based monomers having mono or polysaccharide side chains.

In some embodiments, the hydrophilic uncharged constitutional units include a constitutional unit of Formula (Ha)

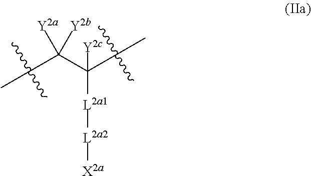

wherein

Y^{2a}, Y^{2b}, and Y^{2c} are each independently selected from H, CN, C₁₋₁₀ alkyl, C₂₋₁₀ alkynyl, C₁₋₁₀ heteroalkyl, cycloalkyl, heterocycloalkyl, aryl, and heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L^{2a1} is absent, C₁₋₁₀ alkylene, C(O), C(O)OC₁₋₁₀ alkylene, C(O)O(CR^{1a}R^{2a})_{q}, C(O)NR^{3a}(CR^{1a}R^{2a})_{q}; (CR^{1a}R^{2a})ₚC(O)O(CR^{1a}R^{2a})_{q}, or (CR^{1a}R^{2a})ₚC(O)NR^{3a}(CR^{1a}R^{2a})_{q};

L^{2a2} is absent, or C(O);

X^{2a} is [O(CH₂)ₘ₁]ₘ₂OR^{4a} or C₁₋₆ alkyl optionally substituted with 1, 2, or 3 OH;

R^{1a} and R^{2a} are each independently H, halo, C₁₋₁₀ alkyl, or C₁₋₁₀ alkyl;

R^{3a} is H, C₁₋₆ alkyl, C₂₋₆ alkenyl, or C₂₋₆ alkynyl;

R^{4a} is H or C₁₋₆ alkyl;

m1 is 1, 2, 3, or 4;

m2 is 5 to 50;

p is 0, 1, 2, 3, or 4; and

q is 0, 1, 2, 3, or 4.

In some embodiments, the hydrophilic uncharged constitutional units of the particle-stabilizing random copolymer have poly(alkylene glycol) side chains, which in turn, has at least 5 (e.g., at least 6, at least 10, at least 20, at least 30, at least 40, or at least 50) and/or at most 100 (e.g., at most 50, at most 40, at most 30, at most 20, at most 10, or at most 6) alkylene glycol constitutional units. For example, the hydrophilic uncharged constitutional units have poly(ethylene glycol) side chains having at least 5 (e.g., at least 6, at least 10, at least 20, at least 30, at least 40, or at least 50) and/or at most 100 (e.g., at most 50, at most 40, at most 30, at most 20, at most 10, or at most 6) ethylene glycol constitutional units. The poly(alkylene glycol) side chain can have a molecular weight of 1000 Daltons or more (e.g., 2000 Da or more, 3000 Da or more, 4000 or more, 5000 or more, or 7000 or more) and/or 10 kDa or less (e.g., 7000 Da or less, 5000 Da or less, 4000 Da or less, 3000 Da or less, or 2000 Da or less). The integration of large PEG-containing monomers (e.g., 1-5 kDa) into the stabilizing polymer can impart significant biocompatibility as well as improved pharmacokinetic properties.

In some embodiments, the hydrophilic uncharged constitutional unit is

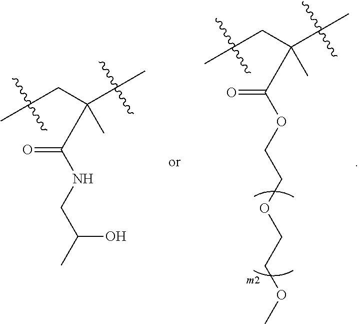

In some embodiments, Y^{2a}, Y^{2b}, and Y^{2c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{2a}, Y^{2b}, and Y^{2c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{2a}, Y^{2b}, and Y^{2c} are each independently selected from H, and C₁₋₁₀ alkyl.

In some embodiments, Y^{2a}, Y^{2b}, and Y^{2c} are each independently selected from H, methyl, ethyl, and propyl.

In some embodiments, Y^{2a}, Y^{2b}, and Y^{2c} are each independently selected from H and methyl.

In some embodiments, L^{2a1} is absent, C₁₋₁₀ alkylene, C(O)OC₁₋₁₀ alkylene, or C(O)O(CR^{1a}R^{2a})_{q}.

In some embodiments, L^{2a1} is absent, C₁₋₁₀ alkylene, or C(O)OC₁₋₁₀ alkylene.

In some embodiments, L^{2a2} is absent.

In some embodiments, L^{2a2} is C(O).

In some embodiments, X^{2a} is [O(CH₂)ₘ₁]ₘ₂OR^{4a}.

In some embodiments, X^{2a} is C₁₋₆ alkyl optionally substituted with 1, 2, or 3 OH.

In some embodiments, R^{1a} and R^{2a} are each independently H or C₁₋₁₀ alkyl.

In some embodiments, R^{1a} and R^{2a} are each independently H or C₁₋₄ alkyl.

In some embodiments, R^{1a} is H or C₁₋₆ alkyl.

In some embodiments, R^{4a} is C₁₋₆ alkyl.

In some embodiments, R^{4a} is methyl.

In some embodiments, R^{4a} is H.

m1 is 1, 2, 3, or 4;

m2 is 5 to 50;

In some embodiments, m1 is 2.

In some embodiments, m2 is 10 to 40 (e.g., 10 to 30, or 10 to 20).

In some embodiments, p is 0.

In some embodiments, p is 1, 2, 3, or 4.

In some embodiments, q is 0.

In some embodiments, q is 1, 2, 3, or 4.

In some embodiments, the hydrophilic uncharged constitutional units include a constitutional unit of Formula (IIb)

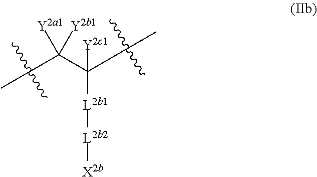

wherein

Y^{2a1}, Y^{2b1}, and Y^{2c1} are each independently selected from H, CN, C₁₋₁₀ alkyl, C₂₋₁₀ alkynyl, C₁₋₁₀ heteroalkyl, cycloalkyl, heterocycloalkyl, aryl, and heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L^{2b1} is absent, C₁₋₁₀ alkylene, C(O), C(O)OC₁₋₁₀ alkylene, C(O)O(CR^{1b}R^{2b})_{q}, C(O)NR^{3b}(CR^{1b}R^{2b})_{q}; (CR^{1b}R^{2b})ₚC(O)O(CR^{1b}R^{2b})_{q}, or (CR^{1b}R^{2b})_{P}C(O)NR^{3b}(CR^{1b}R^{2b})_{q};

L^{2b2} is absent or C(O);

X^{2b} is a polysaccharide;

R^{1b} and R^{2b} are each independently H, halo, C₁₋₁₀ alkyl, or C₁₋₁₀ haloalkyl;

R^{3b} is H, C₁₋₆ alkyl, C₂₋₆ alkenyl, or C₂₋₆ alkynyl;

p is 0, 1, 2, 3, or 4; and

q is 0, 1, 2, 3, or 4.

In some embodiments, the polysaccharide is covalently bound to the constitutional unit of Formula (IIb) via an oxygen, sulfur, nitrogen, or carbon atom. In some embodiments, the polysaccharide has at least 5 (e.g., at least 7, at least 10, at least 15, at least 20, at least 25, at least 30, or at least 50) and/or at most 100 (e.g., at most 50, at most 30, at most 25, at most 20, at most 15, at most 10, or at most 7) saccharide units.

In some embodiments, the hydrophilic uncharged constitutional units of the particle-stabilizing random copolymer have polysaccharide side chains accounting for 10 percent or more (e.g., 15 percent or more, 20 percent or more, or 30 percent or more) and/or 50 percent or less (e.g., 30 percent or less, 20 percent or less, or 15 percent or less) saccharide by mass of a given random copolymer.

In some embodiments, Y^{2a1}, Y^{2b1}, and Y^{2c1} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{2a1}, Y^{2b1}, and Y^{2c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{2a1} Y^{2b1}, and Y^{2c1} are each independently selected from H, and C₁₋₁₀ alkyl.

In some embodiments, Y^{2a1}, Y^{2b1}, and Y^{2c1} are each independently selected from H, methyl, ethyl, and propyl.

In some embodiments, Y^{2a1}, Y^{2b1}, and Y^{2c1} are each independently selected from H and methyl.

In some embodiments, L^{2b1} is absent, C₁₋₁₀ alkylene, C(O)OC₁₋₁₀ alkylene, or C(O)O(CR^{1b}R^{2b})_{q}.

In some embodiments, L^{2b1} is absent, C₁₋₁₀ alkylene, or C(O)OC₁₋₁₀ alkylene.

In some embodiments, L^{2b2} is absent.

In some embodiments, L^{2b2} is C(O).

In some embodiments, R^{1b} and R^{2b} are each independently H or C₁₋₁₀ alkyl.

In some embodiments, R^{1b} and R^{2b} are each independently H or C₁₋₄ alkyl.

In some embodiments, R^{3b} is H or C₁₋₆ alkyl.

In some embodiments, p is 0.

In some embodiments, p is 1, 2, 3, or 4.

In some embodiments, q is 0.

In some embodiments, q is 1, 2, 3, or 4.

In some embodiments, the particle-stabilizing random copolymer includes anionic constitutional units of Formula (IV):

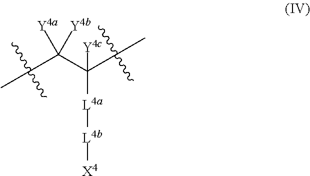

wherein

Y^{4a}, Y^{4b}, and Y^{4c} are each independently selected from H, CN, C₁₋₁₀ alkyl, C₂₋₁₀ alkynyl, C₁₋₁₀ heteroalkyl, cycloalkyl, heterocycloalkyl, aryl, and heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L^{4a} is selected from absent, C₁₋₁₀ alkylene, C(O)O, C(O)OC₁₋₁₀ alkylene, C(O)NR^{3d}, C(O)O(CR^{1d}R^{2d})_{q}, C(O)NR^{3d}(CR^{1d}R^{2d})_{q}, C(O)O(CR^{1d}R^{2d})_{q}C(O)O, C(O)NR^{3d}(CR^{1d}R^{2d})_{q}C(O)O;

L^{4b} is selected from absent, C₁₋₆ alkylene, C₂₋₆ alkenylene, and arylene, and wherein said C₁₋₆ alkylene, C₂₋₆ alkenylene, or arylene is optionally substituted with 1, 2, 3, or 4 substituents independently selected from halo and COOH;

X⁴ is COOR⁵ or SO₃R⁵;

R^{1d} and R^{2d} are each independently H, halo, C₁₋₁₀ alkyl, or C₁₋₁₀ haloalkyl, or

R^{1d} and R^{2d} together with the carbon to which they are attached form an arylene optionally substituted with 1, 2, 3, or 4 halo;

R^{3d} is H, C₁₋₆ alkyl, C₂₋₆ alkenyl, or C₂₋₆ alkynyl;

R⁵ is H or C₁₋₆ alkyl; and

q is 0, 1, 2, 3, 4, 5, 6, 7, 8, 9, or 10.

In some embodiments, Y^{4a}, Y^{4b}, and Y^{4c} are each independently selected from H, C₁₋₁₀ alkyl, C₁₋₁₀ heteroalkyl, wherein said C₁₋₁₀ alkyl, or C₁₋₁₀ hetero alkyl is optionally substituted with 1, 2, 3, or 4 substituents independently selected from halo and C₁₋₁₀ alkyl.

In some embodiments, Y^{4a}, Y^{4b}, and Y^{4c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{4a}, Y^{4b}, and Y^{4c} are each independently selected from H, and C₁₋₁₀ alkyl.

In some embodiments, Y^{4a}, Y^{4b}, and Y^{4c} are each independently selected from H, methyl, ethyl, and propyl.

In some embodiments, Y^{4a}, Y^{4b}, and Y^{4c} are each independently selected from H and methyl.

In some embodiments, L^{4a} is absent, C₁₋₁₀ alkylene, C(O)O, C(O)NR^{3d}, C(O)O(CR^{1d}R^{2d})_{q}, or C(O)NR^{3d}(CR^{1d}R^{2d})_{q}.

In some embodiments, L^{4a} is absent, C(O)O, C(O)NR^{3d}, C(O)O(CR^{1d}R^{2d})_{q}, or C(O)NR^{3d}(CR^{1d}R^{2d})_{q}.

In some embodiments, L^{4b} is selected from absent, C₁₋₆ alkylene, and arylene, wherein said C₁₋₆ alkylene or arylene is optionally substituted with 1, 2, 3, or 4 substituents independently selected from halo and COOH;

In some embodiments, L^{4b} is selected from absent, C₁₋₆ alkylene, and arylene.

In some embodiments, L^{4b} is absent.

In some embodiments, X⁴ is COOR⁵.

In some embodiments, X⁴ is or SO₃R⁵.

In some embodiments, R^{1d} and R^{2d} are each independently H or C₁₋₁₀ alkyl.

In some embodiments, R^{1d} and R^{2d} are each independently H or C₁₋₄ alkyl.

In some embodiments, R^{3d} is H or C₁₋₆ alkyl.

In some embodiments, R⁵ is H.

In some embodiments, R⁵ is t-butyl.

In some embodiments, q is 0, 1, 2, 3, or 4.

In some embodiments, q is 0.

In some embodiments, q is 1, 2, 3, or 4.

In some embodiments, the anionic constitutional unit is selected from

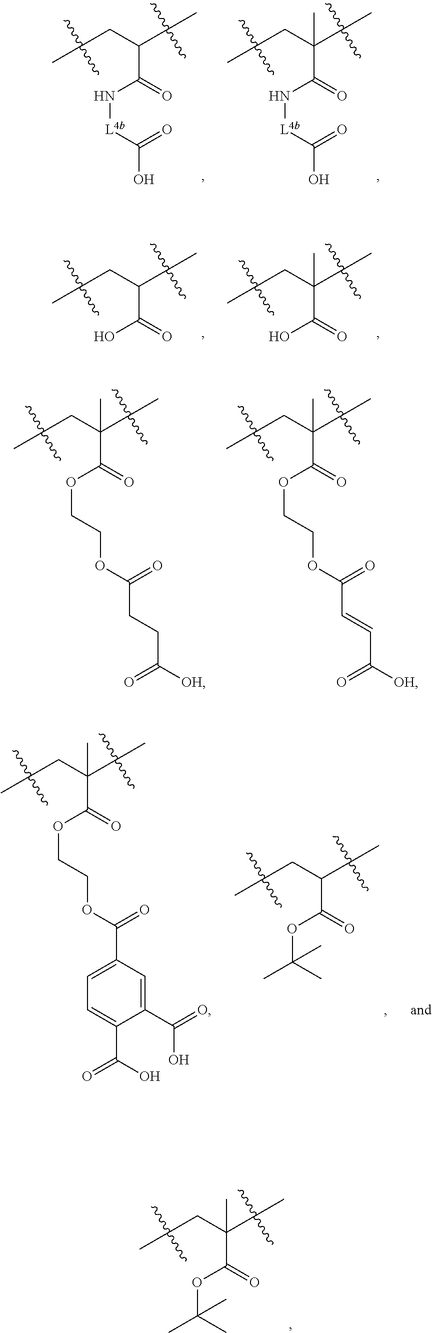

wherein L^{4b} is selected from absent, C₁₋₆ alkylene, C₂₋₆ alkenylene, and arylene, and wherein said C₁₋₆ alkylene, C₂₋₆ alkenylene, or arylene is optionally substituted with 1, 2, 3, or 4 substituents independently selected from halo and COOH.

In some embodiments, the anionic constitutional unit is

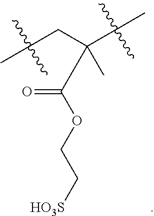 [SMILES: *CC(*)(C)C(=O)OCCS(=O)(=O)O]

In some embodiments, the particle-stabilizing random copolymer includes cationic constitutional units of Formula (V)

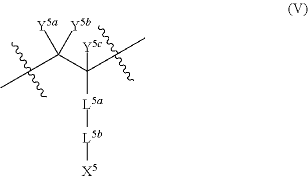

wherein

Y^{5a}, Y^{5b}, and Y^{5c} are each independently selected from H, —CN, C₁₋₁₀ alkyl, C₂₋₁₀ alkynyl, C₁₋₁₀ heteroalkyl, cycloalkyl, heterocycloalkyl, aryl and heteroaryl, wherein said C₁₋₁₀ alkyl, C₂₋₁₀ alkynyl, C₁₋₁₀ heteroalkyl, cycloalkyl, heterocycloalkyl, aryl and heteroaryl is optionally substituted with 1, 2, 3, or 4 substituents independently selected from halo, C₁₋₁₀ alkyl, CN, NO₂, and OH;

L^{5a} is selected from absent, C₁₋₆ alkylene, C₁₋₁₀ alkylene, C(O), C(O)O, C(O)OC₁₋₁₀ alkylene, C(O)NR^{3e}, C(O)O(CR^{1e}R^{2e})_{q}, C(O)NR^{3e}(CR^{1e}R^{2e})_{q}, C(O)O(CR^{1e}R^{2e})_{q}C(O)O, C(O)NR^{3e}(CR^{1e}R^{2e})_{q}C(O)O;

L^{5b} is selected from absent, C₁₋₆ alkylene, C₂₋₆ alkenylene, and arylene, and wherein said C₁₋₆ alkylene, C₂₋₆ alkenylene, or arylene is optionally substituted with 1, 2, 3, or 4 substituents independently selected from halo and COOH;

X⁵ is NR⁶R⁷ or N⁺R⁶R⁷R⁸,

R^{1e} and R^{2e} are each independently H, halo, C₁₋₁₀ alkyl, or C₁₋₁₀ haloalkyl, or

R^{1e} and R^{2e} together with the carbon to which they are attached form an arylene optionally substituted with 1, 2, 3, or 4 halo;

R^{3e} is H, C₁₋₆ alkyl, C₂₋₆ alkenyl, or C₂₋₆ alkynyl;

R⁶, R⁷, and R⁸ are each independently selected from H and C₁₋₆ alkyl; and

q is 0, 1, 2, 3, 4, 5, 6, 7, 8, 9, or 10.

In some embodiments, Y^{5a}, Y^{5b}, and Y^{5c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{5a}, Y^{5b}, and Y^{5c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{5a}, Y^{5b}, and Y^{5c} are each independently selected from H, and C₁₋₁₀ alkyl.

In some embodiments, Y^{5a}, Y^{5b}, and Y^{5c} are each independently selected from H, methyl, ethyl, and propyl.

In some embodiments, Y^{5a}, Y^{5b}, and Y^{5c} are each independently selected from H and methyl.

In some embodiments, L^{5a} is absent, C₁₋₁₀ alkylene, C(O)O, C(O)NR^{3e}, C(O)O(CR^{1e}R^{2e})_{q}, or C(O)NR^{3e}(CR^{1e}R^{2e})_{q}.

In some embodiments, L^{5a} is L^{4a} absent, C(O)O, C(O)NR^{3e}, C(O)O(CR^{1e}R^{2e})_{q}, or C(O)NR^{3e}(CR^{1e}R^{2e})_{q}.

In some embodiments, L^{5b} is selected from absent, C₁₋₆ alkylene, and arylene, wherein said C₁₋₆ alkylene or arylene is optionally substituted with 1, 2, 3, or 4 substituents independently selected from halo and COOH.

In some embodiments, L^{5b} is selected from absent, C₁₋₆ alkylene, and arylene.

In some embodiments, L^{5b} is absent.

In some embodiments, X⁵ is NR⁶R⁷.

In some embodiments, X⁵ is N⁺R⁶R⁷R⁸.

In some embodiments, R^{1e} and R^{2e} are each independently H or C₁₋₁₀ alkyl.

In some embodiments, R^{1e} and R^{2e} are each independently H or C₁₋₄ alkyl.

In some embodiments, R^{3e} is H or C₁₋₆ alkyl.

In some embodiments, R⁶, R⁷, and R⁸ are each independently H or methyl.

In some embodiments, q is 0, 1, 2, 3, or 4.

In some embodiments, q is 0.

In some embodiments, q is 1, 2, 3, or 4.

In some embodiments, the cationic constitutional unit is selected from

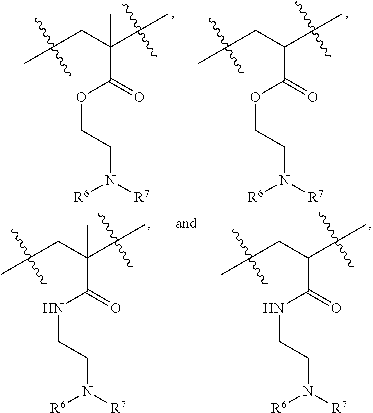

wherein R⁶ and R⁷ are each independently selected from H and C₁₋₆ alkyl.

In some embodiments, the cationic constitutional unit is

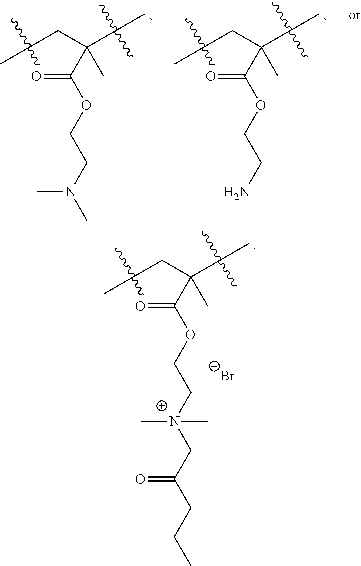 [SMILES: *CC(*)(C)C(=O)OCCN(C)C ; *CC(*)(C)C(=O)OCCN ; *CC(*)(C)C(=O)OCC[N+](C)(C)CC(=O)CCC ; [Br-]]

In some embodiments, the cationic constitutional unit is

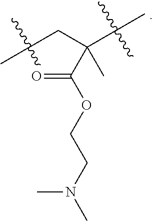 [SMILES: *CC(*)(C)C(=O)OCCN(C)C]

In some embodiments, the constitutional units of the particle-stabilizing random copolymer can be both cationic and anionic (i.e., zwitterionic) and can be formed, for example, of betaine and ampholyte monomers (e.g., carboxybetaines, sulfobetains, or phosphobetaines). For example, the zwitterionic constitutional units can be

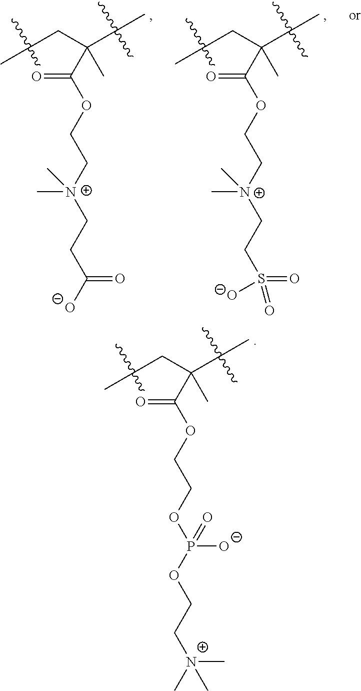 [SMILES: *CC(*)(C)C(=O)OCC[N+](C)(C)CCC(=O)[O-] ; *CC(*)(C)C(=O)OCC[N+](C)(C)CCS(=O)(=O)[O-] ; *CC(*)(C)C(=O)OCCOP(=O)([O-])OCC[N+](C)(C)C]

In some embodiments, the particle-stabilizing random copolymer includes one or more of cationic constitutional units (e.g., formed of N,N-dimethylaminoethyl methacrylate) and anionic constitutional units (e.g., formed of methacrylic acid monomers). Some examples of zwitterionic monomers, cationic monomers, and anionic monomers are provided below.

In some embodiments, the particle-stabilizing random copolymer includes a targeting agent-containing constitutional unit of Formula (III):

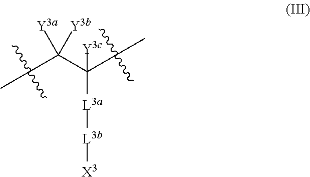

wherein

Y^{3a}, Y^{3b}, and Y^{3c} are each independently selected from H, —CN, C₁₋₁₀ alkyl, C₂₋₁₀ alkynyl, C₁₋₁₀ heteroalkyl, cycloalkyl, heterocycloalkyl, aryl and heteroaryl, wherein said C₁₋₁₀ alkyl, C₂₋₁₀ alkynyl, C₁₋₁₀ heteroalkyl, cycloalkyl, heterocycloalkyl, aryl and heteroaryl is optionally substituted with 1, 2, 3, or 4 substituents independently selected from halo, C₁₋₁₀ alkyl, CN, NO₂, and OH;

L^{3a} is absent, C₁₋₁₀ alkylene, C(O), C(O)OC₁₋₁₀ alkylene, C(O)O(CR^{1c}R^{2c})_{q}, C(O)NR^{3c}(CR^{1c}R^{2c})_{q}; (CR^{1c}R^{2c})ₚC(O)O(CR^{1c}R^{2c})_{q}, or (CR^{1c}R^{2c})ₚC(O)NR^{3c}(CR^{1c}R^{2c})_{q};

L^{3b} is absent, C(O), C(O)O, or C(O)NR^{3c};

X³ is a targeting agent;

R^{1c} and R^{2c} are each independently H, halo, C₁₋₁₀ alkyl, or C₁₋₁₀ haloalkyl;

R^{1c} is H, C₁₋₆ alkyl, C₂₋₆ alkenyl, or C₂₋₆ alkynyl;

p is 0, 1, 2, 3, or 4; and

q is 0, 1, 2, 3, or 4.

In some embodiments, the targeting agent is protein, an antibody, a peptide, a polysaccharide, or a vitamin. For example the targeting agent can be transferrin, a glycan, biotin, folic acid, or a vitamin B. The targeting agent selectively induces binding and/or internalization of the particle assembly or the particle-stabilizing random copolymer into a cell.

In some embodiments, Y^{3a}, Y^{3b}, and Y^{3c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{3a}, Y^{3b}, and Y^{3c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{3a}, Y^{3b}, and Y^{3c} are each independently selected from H, and C₁₋₁₀ alkyl.

In some embodiments, Y^{3a}, Y^{3b}, and Y^{3c} are each independently selected from H, methyl, ethyl, and propyl.

In some embodiments, Y^{3a}, Y^{3b}, and Y^{3c} are each independently selected from H and methyl.

In some embodiments, L^{3a} is absent, C₁₋₁₀ alkylene, C(O)O, C(O)NR^{3c}, C(O)O(CR^{1c}R^{2c})_{q}, or C(O)NR^{3c}(CR^{1c}R^{2c})_{q}.

In some embodiments, L^{3a} is absent, C(O)O, C(O)NR^{3c}, C(O)O(CR^{1c}R^{2c})_{q}, or C(O)NR^{3c}(CR^{1c}R^{2c})_{q}.

In some embodiments, L^{3b} is C(O), C(O)O, or C(O)NR^{3c}.

In some embodiments, L^{3b} is absent.

In some embodiments, L^{3b} is C(O)O or C(O)NR^{3c}.

In some embodiments, R^{1c} and R^{2c} are each independently H or C₁₋₁₀ alkyl.

In some embodiments, R^{1c} and R^{2c} are each independently H or C₁₋₄ alkyl.

In some embodiments, R^{3c} is H or C₁₋₆ alkyl.

In some embodiments, p is 0.

In some embodiments, p is 1, 2, 3, or 4.

In some embodiments, q is 0.

In some embodiments, q is 1, 2, 3, or 4.

The various constitutional units can be present in the particle-stabilizing random copolymer in various proportions. For example, in the particle-stabilizing random copolymer, constitutional units (I) can be present in an amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less); constitutional units (IIa) and/or (IIb) can be present in a cumulative amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less); constitutional units (III) can be present in an amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less); and constitutional units (IV) and/or (V) can be present in a cumulative amount of from 0 to 80 mole percent to 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less), so long as the total mole percent of constitutional units (I), (IIa) and/or (IIb), (III), and (IV) and/or (V) adds to 100 mole percent. In some embodiments, each of constitutional units (I), (IIa), (IIb), (III), (IV), and/or (V) can independently be present in an amount of greater than 0 mole percent.

The particle-stabilizing random copolymer can span a wide range of compositions, depending on the constituent units and the desired particle assembly morphology. For example, for example, large polyethylene glycol methacrylates (MW>1000 Da) are effective at stabilizing copolymers containing significant hydrophobic content even at lower hydrophilic comonomer ratios.

For example, in one embodiment, the particle-stabilizing random copolymer includes hydrophobic constitutional units having fatty acid ester side chains (e.g., lauryl methacrylate), hydrophilic constitutional units having polyalkylene glycol side chains (e.g., polyethylene glycol side chains from methacrylate “PEGMA” monomers), and constitutional units having glycan targeting groups.

In some embodiments, the particle-stabilizing random copolymer is a copolymer of Formula (A), synthesized according to Scheme 1, below.

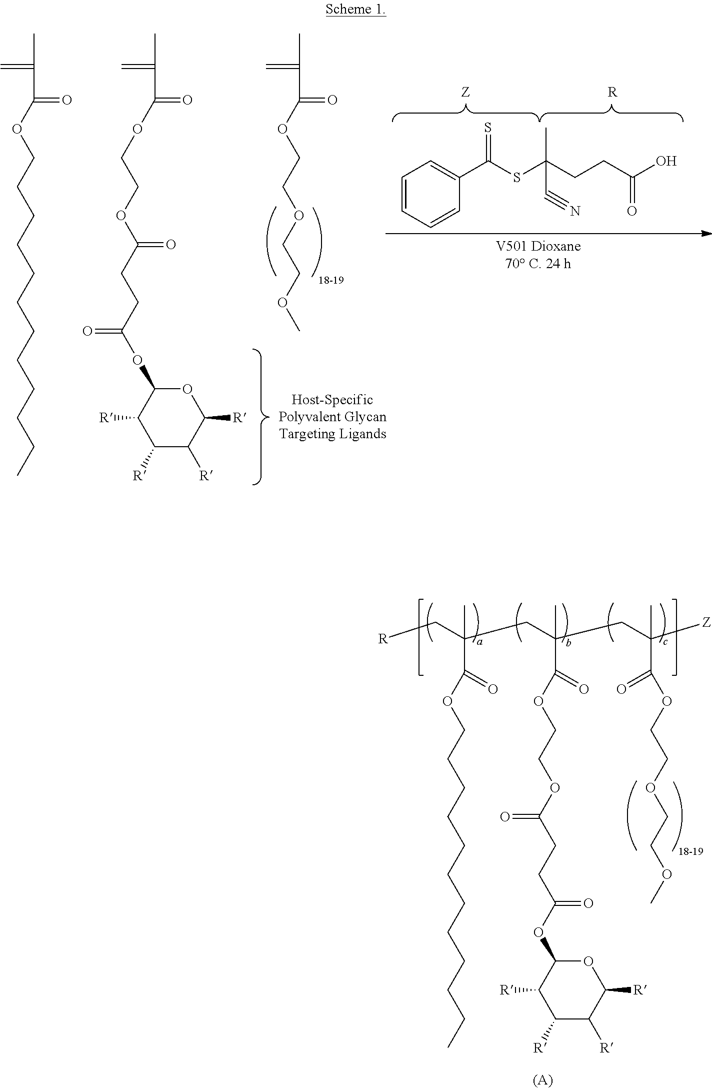

### Core Random Copolymer

The core random copolymer can be relatively hydrophobic and can be formed of any combination of the vinyl monomers that phase separate from physiological solutions in their polymerized form. For example, the core random copolymer can be formed of methacrylates and acrylamide monomers that render the core random copolymer relatively hydrophobic compared to the particle-stabilizing random copolymer.

In some embodiment, the core random copolymer includes hydrophobic uncharged constitutional units. The hydrophobic uncharged constitutional units of the core random copolymer may have some hydrophilic character but is relatively hydrophobic compared to the particle-stabilizing random copolymer with which the core random copolymer is paired. For example, when the hydrophilic constitutional units of the particle-stabilizing random copolymer have 10 or more ethylene glycol constitutional units, the hydrophobic uncharged constitutional units of the core random copolymer can include poly(ethylene glycol) side chains having at most 4 ethylene glycol constitutional units.

In some embodiments, the core random copolymer includes a hydrophobic uncharged constitutional unit of Formula (I), as described above.

In some embodiments, the core random copolymer includes a hydrophobic uncharged constitutional unit of Formula (VI):

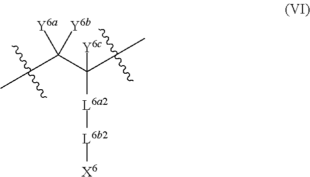

wherein

Y^{6a}, Y^{6b}, and Y^{6c} are each independently selected from H, CN, C₁₋₁₀ alkyl, C₂₋₁₀ alkynyl, C₁₋₁₀ heteroalkyl, cycloalkyl, heterocycloalkyl, aryl, and heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L^{6a1} is absent, C₁₋₁₀ alkylene, C(O), C(O)OC₁₋₁₀alkylene, C(O)O(CR^{1f}R^{2f})_{q}, C(O)NR^{3f}(CR^{1f}R^{2f})_{q}; (CR^{1f}R^{2f})ₚC(O)O(CR^{1f}R^{2f})_{q}, or (CR^{1f}R^{2f})ₚC(O)NR^{3f}(CR^{1f}R^{2f})_{q};

L^{6a2} is absent or C(O);

X^{6a} is H or [O(CH₂)ₘ₁]ₘ₂OR^{4f};

R^{1f} and R^{2f} are each independently H, halo, or C₁₋₆ alkyl;

R^{3f} is H, C₁₋₆ alkyl, C₂₋₆ alkenyl, or C₂₋₆ alkynyl;

R^{4f} is C₁₋₂₄ alkyl;

m1 is 1, 2, 3, or 4;

m2 is 0 to 4;

p is 0, 1, 2, 3, or 4; and

q is 0, 1, 2, 3, or 4.

In some embodiments, Y^{6a}, Y^{6b}, and Y^{6c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{6a}, Y^{6b}, and Y^{6c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{6a}, Y^{6b}, and Y^{6c} are each independently selected from H, and C₁₋₁₀ alkyl.

In some embodiments, Y^{6a}, Y^{6b}, and Y^{6c} are each independently selected from H, methyl, ethyl, and propyl.

In some embodiments, Y^{6a}, Y^{6b}, and Y^{6c} are each independently selected from H and methyl.

In some embodiments, L^{6a1} is absent, C₁₋₁₀ alkylene, C(O)OC₁₋₁₀ alkylene, or C(O)O(CR^{1f}R^{2f})_{q}.

In some embodiments, L^{6a1} is absent, C₁₋₁₀ alkylene, or C(O)OC₁₋₁₀ alkylene.

In some embodiments, L^{6a2} is absent.

In some embodiments, L^{6a2} is C(O).

In some embodiments, X^{6a} [O(CH₂)ₘ₁]ₘ₂OR^{4f}.

In some embodiments, R^{1f} and R^{2f} are each independently H or C₁₋₁₀ alkyl.

In some embodiments, R^{1f} and R^{2f} are each independently H or C₁₋₄ alkyl.

In some embodiment, R^{3f} is H or C₁₋₆ alkyl.

In some embodiments, R^{4f} is C₈₋₂₄ alkyl (e.g., C₁₂₋₂₄ alkyl, C₁₂₋₂₀ alkyl, or C₁₂ alkyl).

In some embodiments, m1 is 2.

In some embodiments, m2 is 1 or 2.

In some embodiments, p is 0.

In some embodiments, p is 1, 2, 3, or 4.

In some embodiments, q is 0.

In some embodiments, q is 1, 2, 3, or 4.

In some embodiments, the core random copolymer includes anionic constitutional units of Formula (IV), as described above.

In some embodiments, the anionic constitutional unit of the core random copolymer is selected from

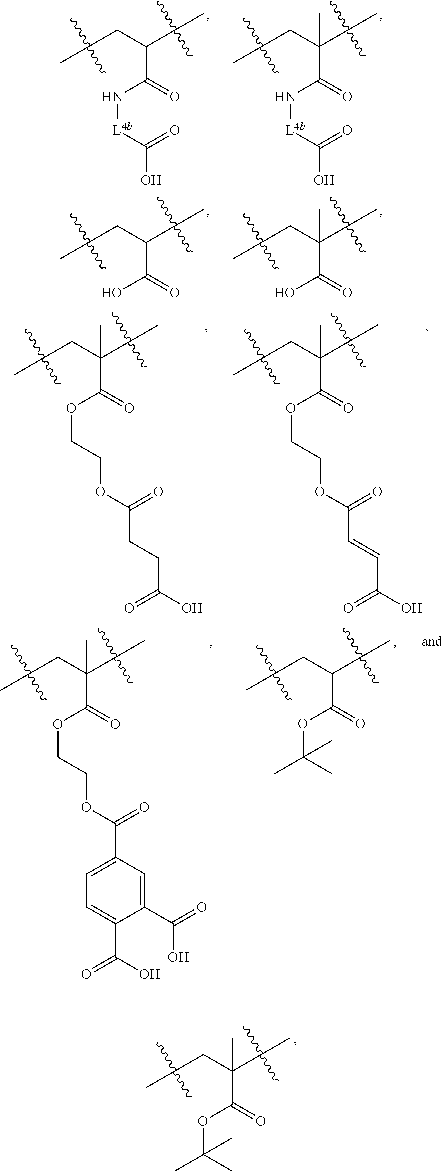

wherein L^{4b} is selected from absent, C₁₋₆ alkylene, C₂₋₆ alkenylene, and arylene, and wherein said C₁₋₆ alkylene, C₂₋₆ alkenylene, or arylene is optionally substituted with 1, 2, 3, or 4 substituents independently selected from halo and COOH.

In some embodiments, the core random copolymer includes cationic constitutional units of Formula (V), as described above.

In some embodiments, the cationic constitutional unit of the core random copolymer is selected from

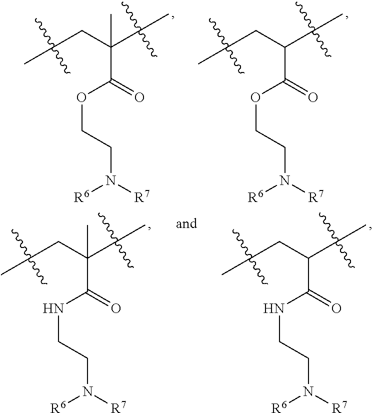

wherein R⁶ and R⁷ are each independently selected from H and C₁₋₆ alkyl.

In some embodiments, the cationic constitutional unit is

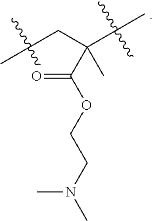 [SMILES: *CC(*)(C)C(=O)OCCN(C)C]

In some embodiments, when the core random copolymer includes both cationic and anionic constitutional units, the ratio of the cationic to anionic constitutional units can be relatively equal.

In some embodiments, the particle-stabilizing random copolymer includes a therapeutic agent. For example, the particle-stabilizing random copolymer can include a including a covalently-bound therapeutic agent-containing constitutional unit of Formula (VII):

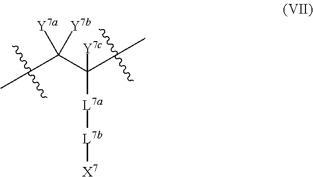

wherein

Y^{7a}, Y^{7b}, and Y^{7c} are each independently selected from H, CN, C₁₋₁₀ alkyl, C₂₋₁₀ alkynyl, C₁₋₁₀ heteroalkyl, cycloalkyl, heterocycloalkyl, aryl, and heteroaryl, wherein said C₁₋₁₀ alkyl, C₂₋₁₀ alkynyl, C₁₋₁₀ heteroalkyl, cycloalkyl, heterocycloalkyl, aryl, or heteroaryl is optionally substituted with 1, 2, 3, or 4 substituents independently selected from halo, C₁₋₁₀ alkyl, CN, NO₂, and OH;

L^{7a} is selected from absent, C₁₋₁₀ alkylene, C(O), C(O)OC₁₋₁₀alkylene, C(O)O(CR^{1g}R^{2g})_{q}, C(O)NR^{3g}(CR^{1g}R^{2g})_{q}; (CR^{1g}R^{2g})ₚC(O)O(CR^{1g}R^{2c})_{q}, (CR^{1g}R^{2g})ₚC(O)NR^{3g}(CR^{1g}R^{2g})_{q}, C(O)O(CR^{1g}R^{2g})ₚC(O)O(CR^{1g}R^{2g})_{q}, C(O)O(CR^{1g}R^{2g})ₚC(O)NR^{3g}(CR^{1g}R^{2g})_{q}, C(O)NR^{3g} (CR^{1g}R^{2g})ₚC(O)NR^{3g}(CR^{1g}R^{2g})_{q}, C(O)NR^{3g}(CR^{1g}R^{2g})ₚC(O)O(CR^{1g}R^{2g})_{q};

L^{7b} is absent, C(O), C(O)O, or C(O)NR^{3c};

X⁷ is a therapeutic agent;

R^{1g} and R^{2g} are each H, halo, C₁₋₁₀ alkyl, or C₁₋₁₀ alkyl;

R^{3g} is H, C₁₋₆ alkyl, C₂₋₆ alkenyl, or C₂₋₆ alkynyl;

p is 0, 1, 2, 3, or 4; and

q is 0, 1, 2, 3, or 4.

Therapeutic agents will be described in greater detail below.

In some embodiments, Y^{7a}, Y^{7b}, and Y^{7c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{7a}, Y^{7b}, and Y^{7c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{7a}, Y^{7b}, and Y^{7c} are each independentl_{y} selected from H, and C₁₋₁₀ alkyl.

In some embodiments, Y^{7a}, Y^{7b}, and Y^{7c} are each independently selected from H, methyl, ethyl, and propyl.

In some embodiments, Y^{7a}, Y^{7b}, and Y^{7c} are each independently selected from H and methyl.

In some embodiments, L^{7a} is absent, C₁₋₁₀ alkylene, C(O)O, C(O)NR^{3g}, C(O)O(CR^{1g}R^{2g})_{q}, or C(O)NR^{3g}(CR^{1g}R^{2g})_{q}.

In some embodiments, L^{7a} is absent, C(O)O, C(O)NR^{3c}, C(O)O(CR^{1g}R^{2g})_{q}, or C(O)NR^{3g}(CR^{1g}R^{2g})_{q}.

In some embodiments, L^{7b} is C(O), C(O)O, or C(O)NR^{3g}.

In some embodiments, L^{7b} is absent.

In some embodiments, L^{7b} is C(O)O or C(O)NR^{3g}.

In some embodiments, R^{1g} and R^{2g} are each independently H or C₁₋₁₀ alkyl.

In some embodiments, R^{1g} and R^{2g} are each independently H or C₁₋₄ alkyl.

In some embodiments, R^{3g} is H or C₁₋₆ alkyl.

In some embodiments, p is 0.

In some embodiments, p is 1, 2, 3, or 4.

In some embodiments, q is 0.

In some embodiments, q is 1, 2, 3, or 4.

In the core random copolymer, the hydrophobic uncharged constitutional unit (Formula (I) and/or (IV)) can be present in an amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less); the cationic constitutional units (Formula (V)) and/or the anionic constitutional units (Formula (IV)) can be present in an amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less); hydrophobic uncharged constitutional unit (Formula (VI)) and the therapeutic agent constitutional unit (Formula (VII)) can be present in an amount of 0 mole percent or more (e.g., 5 mole percent or more, 10 mole percent or more, 25 mole percent or more, 50 mole percent or more, or 75 mole percent or more) and/or 80 mole percent or less (e.g., 75 mole percent or less, 50 mole percent or less, 25 mole percent or less, 10 mole percent or less, or 5 mole percent or less), so long as the total mole percent of constitutional units adds to 100 mole percent. In some embodiments, each of hydrophobic uncharged constitutional unit (Formula (I) and/or (IV)), cationic constitutional units (Formula (V)), anionic constitutional units (Formula (IV)), hydrophobic uncharged constitutional unit (Formula (VI)) and/or the therapeutic agent constitutional unit (Formula (VII)) can be present in an amount of greater than 0 mole percent.

### Polymeric Drug Carriers

The particle-stabilizing random copolymer and the core random copolymer, described above, can each be used as a polymeric drug carrier, when conjugated to a therapeutic agent. The therapeutic agent can be conjugated to the copolymer via a cleavable linkage, such that when the therapeutic agent is released from the copolymer upon exposure to physiological conditions (e.g., cleaved from the copolymer by an enzyme or water). The therapeutic agent can be released from the copolymer over an extended period of time. For example, the therapeutic agent can be released from the copolymer over a period of 1 hour (e.g., 12 hours, 24 hours, 48 hours, 100 hours, 200 hours, 400 hours, 600 hours, or 800 hours) to 900 hours (e.g., 800 hours, 600 hours, 400 hours, 200 hours, 100 hours, 48 hours, 24 hours, or 12 hours). The therapeutic agent can be released in a linear manner, or the rate of release can increase or decrease over time.

When the particle-stabilizing random copolymer is used as a carrier for a therapeutic agent, the particle-stabilizing random copolymer can be formed of the same constitutional units as described above, but may or may not include a constitutional unit that is conjugated to a targeting agent. For example, the random copolymer carrier can include (a) hydrophobic uncharged constitutional units, cationic constitutional units, or anionic constitutional units, or any combination thereof, (b) hydrophilic constitutional units, (c) constitutional units including a covalently-bound antibiotic agent or kinase inhibitor, and optionally (d) constitutional units including a targeting agent, wherein the antibiotic agent or kinase inhibitor is cleavable from the copolymer by an enzyme or water. The hydrophobic uncharged constitutional units, cationic constitutional units, anionic constitutional units, hydrophilic constitutional units, and constitutional units including a targeting agent are as described above.

When the particle-stabilizing random copolymer is used as a drug carrier, the random copolymer can include 0 mole percent or more (e.g., 5 mole percent or more, 10 mole percent or more, 25 mole percent or more, 50 mole percent or more, or 75 mole percent or more) and/or 90 mole percent or less (e.g., 75 mole percent or less, 50 mole percent or less, 25 mole percent or less, 10 mole percent or less, or 5 mole percent or less) of hydrophobic uncharged constitutional units, 0 mole percent or more (e.g., 5 mole percent or more, 10 mole percent or more, 25 mole percent or more, 50 mole percent or more, or 75 mole percent or more) and/or 90 mole percent or less (e.g., 75 mole percent or less, 50 mole percent or less, 25 mole percent or less, 10 mole percent or less, or 5 mole percent or less) of hydrophilic constitutional units, and 0 mole percent or more (e.g., 5 mole percent or more, 10 mole percent or more, 25 mole percent or more, or 40 mole percent or more) and/or 50 mole percent or less (e.g., 40 mole percent or less, 25 mole percent or less, 10 mole percent or less, or 5 mole percent or less) of constitutional units comprising a covalently-bound antibiotic agent or kinase inhibitor. In some embodiments, each of the hydrophobic uncharged constitutional units, the hydrophilic constitutional units, and/or the constitutional units comprising a covalently-bound antibiotic agent or kinase inhibitor can be present in an amount of greater than 0 mole percent. In some embodiments, the molar ratio of hydrophobic to hydrophilic constitutional units is such that the overall random copolymer remains soluble under aqueous conditions. In certain embodiments, depending on the molecular weight of a given hydrophilic constitutional unit, aqueous solubility can be obtained for a polymer even when the polymer contains a small mole percentage of the hydrophilic constitutional unit. For example, PEGMA at 2000 Da can provide aqueous solubility to polymers containing hydrophobic constitutional units at 10 mole percent.

In some embodiments, the random copolymer includes 0 mole percent or more (e.g., 5 mole percent or more, 10 mole percent or more, 25 mole percent or more, or 40 mole percent or more) and/or 45 mole percent or less (e.g., 40 mole percent or less, 25 mole percent or less, 10 mole percent or less, or 5 mole percent or less) of anionic constitutional units, 0 mole percent or more (e.g., 5 mole percent or more, 10 mole percent or more, 25 mole percent or more, or 40 mole percent or more) and/or 45 mole percent or less (e.g., 40 mole percent or less, 25 mole percent or less, 10 mole percent or less, or 5 mole percent or less) of cationic constitutional units, 0 mole percent or more (e.g., 5 mole percent or more, 10 mole percent or more, 25 mole percent or more, or 40 mole percent or more) and/or 50 mole percent or less (e.g., 40 mole percent or less, 25 mole percent or less, 10 mole percent or less, or 5 mole percent or less) of hydrophilic constitutional units, and 0 mole percent or more (e.g., 5 mole percent or more, 10 mole percent or more, 25 mole percent or more, or 40 mole percent or more) and/or 50 mole percent or less (e.g., 40 mole percent or less, 25 mole percent or less, 10 mole percent or less, or 5 mole percent or less) of constitutional units including a covalently bound antibiotic agent or kinase inhibitor. In some embodiments, each of the anionic constitutional units, cationic constitutional units, hydrophilic constitutional units, and/or constitutional units including a covalently bound antibiotic agent or kinase inhibitor can be present in an amount of greater than 0 mole percent. In some embodiments, the random copolymer includes equimolar cationic and anionic constitutional units and from greater than 0 to 50 mole percent constitutional units including a covalently bound antibiotic agent or kinase inhibitor. The cationic and anionic constitutional units are both hydrophilic, and additional hydrophilic residues can be added at any molar ratio, provided that the combination of anionic, cationic, and neutral hydrophilic residues adds up to at least 50 mole percent.

In some embodiments, the random copolymer includes 0 mole percent or more (e.g., 5 mole percent or more, 10 mole percent or more, 25 mole percent or more, 50 mole percent or more, or 60 mole percent or more) and/or 70 mole percent or less (e.g., 60 mole percent or less, 50 mole percent or less, 25 mole percent or less, 10 mole percent or less, or 5 mole percent or less) of anionic constitutional units, 0 mole percent or more (e.g., 5 mole percent or more, 10 mole percent or more, 25 mole percent or more, 50 mole percent or more, or 60 mole percent or more) and/or 70 mole percent or less (e.g., 60 mole percent or less, 50 mole percent or less, 25 mole percent or less, 10 mole percent or less, or 5 mole percent or less) of hydrophilic constitutional units, and 0 mole percent or more (e.g., 5 mole percent or more, 10 mole percent or more, 25 mole percent or more, or 40 mole percent or more) and/or 50 mole percent or less (e.g., 40 mole percent or less, 25 mole percent or less, 10 mole percent or less, or 5 mole percent or less) of constitutional units comprising a covalently bound antibiotic agent or kinase inhibitor. In some embodiments, each of the anionic constitutional units, hydrophilic constitutional units, and/or constitutional units including a covalently bound antibiotic agent or kinase inhibitor can be present in an amount of greater than 0 mole percent.

In some embodiments, the random copolymer includes 0 mole percent or more (e.g., 5 mole percent or more, 10 mole percent or more, 25 mole percent or more, 50 mole percent or more, or 60 mole percent or more) and/or 70 mole percent or less (e.g., 60 mole percent or less, 50 mole percent or less, 25 mole percent or less, 10 mole percent or less, or 5 mole percent or less) of cationic constitutional units, 0 mole percent or more (e.g., 5 mole percent or more, 10 mole percent or more, 25 mole percent or more, 50 mole percent or more, or 60 mole percent or more) and/or 70 mole percent or less (e.g., 60 mole percent or less, 50 mole percent or less, 25 mole percent or less, 10 mole percent or less, or 5 mole percent or less) of hydrophilic constitutional units, and 0 mole percent or more (e.g., 5 mole percent or more, 10 mole percent or more, 25 mole percent or more, or 40 mole percent or more) and/or 50 mole percent or less (e.g., 40 mole percent or less, 25 mole percent or less, 10 mole percent or less, or 5 mole percent or less) of constitutional units comprising a covalently bound antibiotic agent or kinase inhibitor. In some embodiments, each of the cationic constitutional units, hydrophilic constitutional units, and/or constitutional units including a covalently bound antibiotic agent or kinase inhibitor can be present in an amount of greater than 0 mole percent.

When the core random copolymer is used as a carrier for a therapeutic agent, the random copolymer can include (i) cationic constitutional units, anionic constitutional units, or any combination thereof, and/or (ii) hydrophobic uncharged constitutional units, and (iii) constitutional units including a covalently-bound antibiotic agent or kinase inhibitor, wherein the antibiotic agent or kinase inhibitor is cleavable from the copolymer by an enzyme or water. The cationic constitutional units, anionic constitutional units, or hydrophobic uncharged constitutional units are as described above.

When the core random copolymer is used as a drug carrier, the random copolymer includes 0 to 90 mole percent of cationic and/or anionic constitutional units, and/or 0 to 70 mole percent of hydrophobic uncharged constitutional units, and 0 to 50 mole percent of constitutional units comprising a covalently-bound antibiotic agent or kinase inhibitor.

In some embodiments, when the core random copolymer is used as a carrier for a therapeutic agent, the random copolymer includes cationic constitutional units, hydrophobic uncharged constitutional units, and constitutional units comprising a covalently-bound antibiotic agent or kinase inhibitor.

In some embodiments, rather than using a particle-stabilizing random copolymer or a core random copolymer as a polymeric carrier for a therapeutic agent, a polymeric carrier that includes a therapeutic agent-containing constitutional unit and one other type of constitutional unit (anionic, cationic, hydrophobic, or hydrophilic constitutional unit, as described above) can be used.

When the particle-stabilizing random copolymer or the core random copolymer is used as a polymeric drug carrier, the polymeric drug carrier can include constitutional units including a covalently-bound antibiotic agent or kinase inhibitor having Formula (VIII):

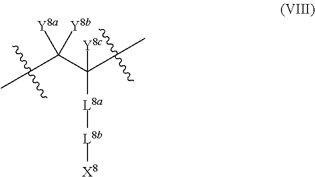

wherein

Y^{8a}, Y^{8b}, and Y^{8c} are each independently selected from H, —CN, C₁₋₁₀ alkyl, C₂₋₁₀ alkynyl, C₁₋₁₀ heteroalkyl, cycloalkyl, heterocycloalkyl, aryl and heteroaryl, wherein said C₁₋₁₀ alkyl, C₂₋₁₀ alkynyl, C₁₋₁₀ heteroalkyl, cycloalkyl, heterocycloalkyl, aryl and heteroaryl is optionally substituted with 1, 2, 3, or 4 substituents independently selected from halo, C₁₋₁₀ alkyl, CN, NO₂, and OH;

L^{8a} is selected from absent, C₁₋₁₀ alkylene, C(O), C(O)OC₁₋₁₀alkylene, C(O)O(CR^{1h}R^{2h})_{q}, C(O)NR^{3h}(CR^{1h}R^{2h})_{q}; (CR^{1h}R^{2h})ₚC(O)O(CR^{1h}R^{2h})_{q}, (CR^{1h}R^{2h})ₚC(O)NR^{3h}(CR^{1h}R^{2h})_{q}, C(O)O(CR^{1h}R^{2h})ₚC(O)O(CR^{1h}R^{2h})_{q}, C(O)O(CR^{1h}R^{2h})ₚC(O)NR^{3h}(CR^{1h}R^{2h})_{q}, C(O)NR^{3h}(CR^{1h}R^{2h})ₚC(O)NR^{3h}(CR^{1h}R^{2h})_{q}, C(O)NR^{3h}(CR^{1h}R^{2h})ₚC(O)O(CR^{1h}R^{2h})_{q};

L^{8b} is absent, C(O), C(O)O, or C(O)NR^{3h};

X⁸ is an antibiotic agent or a kinase inhibitor;

R^{1h} and R^{2h} are each independently H, halo, C₁₋₁₀ alkyl, or C₁₋₁₀ haloalkyl;

R^{3h} is H, C₁₋₆ alkyl, C₂₋₆ alkenyl, or C₂₋₆ alkynyl;

p is 0, 1, 2, 3, or 4; and

q is 0, 1, 2, 3, or 4.

In some embodiments, Y^{8a}, Y^{8b}, and Y^{8c} are each independently selected from H, C₁₋₁₀ alkyl, C₁₋₁₀ heteroalkyl, wherein said C₁₋₁₀ alkyl, or C₁₋₁₀ heteroalkyl is optionally substituted with 1, 2, 3, or 4 substituents independently selected from halo and C₁₋₁₀ alkyl.

In some embodiments, Y^{8a}, Y^{8b}, and Y^{8c} are each independently selected from H, and C₁₋₁₀ alkyl, wherein said C₁₋₁₀ alkyl is optionally substituted with 1, 2, 3, or 4 substituents independently selected from halo and C₁₋₁₀ alkyl.

In some embodiments, Y^{8a}, Y^{8b}, and Y^{8c} are each independently selected from H, and C₁₋₁₀ alkyl.

In some embodiments, Y^{8a}, Y^{8b} and Y^{8c} are each independently selected from H, methyl, ethyl, and propyl.

In some embodiments, Y^{8a}, Y^{8b} and Y^{8c} are each independently selected from H and methyl.

In some embodiments, L^{8a} is absent, C₁₋₁₀ alkylene, C(O)O, C(O)NR^{3h}, C(O)O(CR^{1h}R^{2h})_{q}, or C(O)NR^{3h}(CR^{1h}R^{2h})_{q}.

In some embodiments, L^{8a} is absent, C(O)O, C(O)NR^{3h}, C(O)O(CR^{1h}R^{2h})_{q}, or C(O)NR^{3h}(CR^{1h}R^{2h})_{q}.

In some embodiments, L^{8b} is C(O), C(O)O, or C(O)NR^{3h}.

In some embodiments, L^{8b} is absent.

In some embodiments, L^{8b} is C(O)O or C(O)NR^{3h}.

In some embodiments, R^{1h} and R^{2h} are each independently H or C₁₋₁₀ alkyl.

In some embodiments, R^{1h} and R^{2h} are each independently H or C₁₋₄ alkyl.

In some embodiments, R^{3h} is H or C₁₋₆ alkyl.

In some embodiments, p is 0.

In some embodiments, p is 1, 2, 3, or 4.

In some embodiments, q is 0.

In some embodiments, q is 1, 2, 3, or 4.

In some embodiments, the antibiotic agent or kinase inhibitor has a reactive moiety that can form a covalent bond to the constitutional unit. For example, the antibiotic agent or kinase inhibitor can be linked to the constitutional unit via a hydrazone bond, via an ester bond, or via an amide bond.

### Therapeutic Agent

As described above, the therapeutic agent (e.g., an antibiotic agent or a kinase inhibitor) can be cleaved from the polymer to which it is conjugated to. In some embodiments, the therapeutic agent is modified in such a way that hydrolysis or enzymatic cleavage provides the parent therapeutic agent. In some embodiments, cleavage from the polymer does not provide the original therapeutic agent (prior to conjugation to the polymer), but can release a modified therapeutic agent that can undergo further modification in a physiological environment, such that the modified therapeutic agent can then release the therapeutic agent in an active form at a different rate than the initial cleavage rate. In some embodiments, even though a native therapeutic agent has been modified to facilitate polymeric conjugation, release of the modified therapeutic agent can still provide a therapeutically active molecule. In some embodiments, the therapeutic agent does not need to be removed from the polymer but can still be therapeutically active, so long as it can reach a cytoplasm and be sufficiently spaced apart from the polymeric backbone.

In some embodiments, the therapeutic agent is an antibiotic agent or a kinase inhibitor. Examples of antibiotic agents include amikacin, gentamicin, neomycin, netilmicin, tobramycin, paromomycin, streptomycin, spectinomycin, geldanamycin, herbimycin, rifaximin, loracarbef, mertapenem, doripenem, imipenem, meropenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriazxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin methicillin, nafcillin, oxicillin, penicillin, piperacillin, temocillin, ticarcillin, piperacillin, ticarcillin, bacitracin, colistin, polymyxin B, xacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole, sulfonamidochrysoidine, demeclocycline, doxycycline, minocycline, oxytetracycline, tetracycline, clofazimine, dapsone, capreomycin, cycloserine, ethambutol, ethionamide, isoniazid, pyrazinamide, rifampin, rifabutin, rifapentine, streptomycin, arsphenamine, chloramphenicol, fosfomycin, fusidic acid, metronidazole, mupirocin, platensimycin, quinupristin, thiamphenicol, tigecycline, tinidazole, and trimethoprim. In some embodiments, the antibiotic agent is ciproflaxin, meropenem, doxycycline, and/or ceftazidime.

Examples of kinase inhibitors include, for example, afatinib, axitinib, bevacizumab, bosutinib, cetuximab, crizotinib, dasatinib, erlotinib, fostamatinib, gefitinib, ibrutinib, imatinib, lapatinib, lenvatinib, nilotinib, panitumumab, pazopanib, pegaptanib, ranibizumab, roxolitinib, sorafenib, sunitinib, SU6656, trastuzumab, tofacitinib, vemurafenib. In some embodiments, the kinase inhibitor is dasatinib.

In some embodiments, the therapeutic agent is a chemotherapeutic agent, such as a vinca alkaloid or a taxane. Examples of chemotherapeutic agents include illudin, aminitin, gemcitabine, etoposide, docetaxel, camptothecin, and paclitaxel.

Conjugation of the Therapeutic Agent

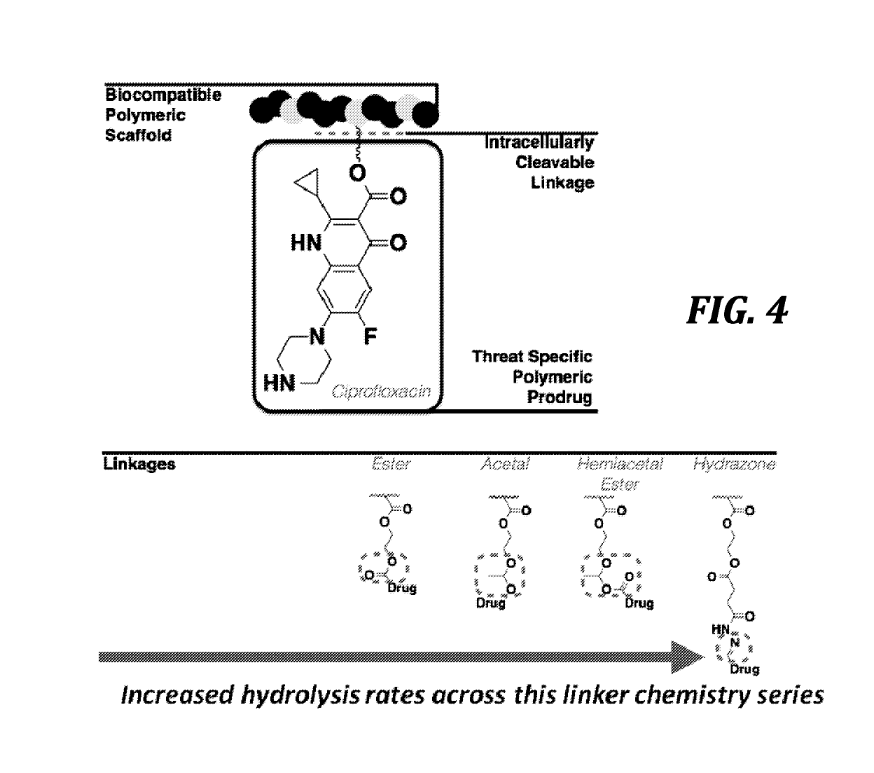

The therapeutic agent can have or can be modified with a reactive functional group that can react with a monomer to be incorporated into a polymer, or with an existing constitutional unit in a polymer. For example, the therapeutic agent can be modified with a COOH, amino, alkylamino, ester, hydrazine, aldehyde, ketone, anhydride, imine, and/or hydroxyl group. Without wishing to be bound by theory, referring to FIG. 4, it is believed that hydrolysis rates can increase as the linker progresses from ester to acetal, to hemiacetal ester, to hydrazone. Methods on how to prepare therapeutic agents with a reactive functional group are known to one skilled in the art of organic synthesis. For example, therapeutic agents containing a carboxylic acid or hydroxyl group can be attached to a monomer using carbodiimide or uronium chemistry catalyzed by DMAP. For monomers that also contain other reactive groups such as amine, protecting groups can be employed. These protecting groups can then be removed prior to polymerization or left in place to facilitate the addition of other potential reactive chemical functionality. For example, t-BOC groups can be cleaved with strong acids such as trifluoroacetic acid while fMOC groups can be removed with organic bases such as piperidine. Protecting groups are described, for example, in Wuts, P. G. M. and Greene, T. W., Greene's Protective Groups in Organic Synthesis, Wiley-Interscience, 4^{th} Edition, 2006, herein incorporated by reference in its entirety.

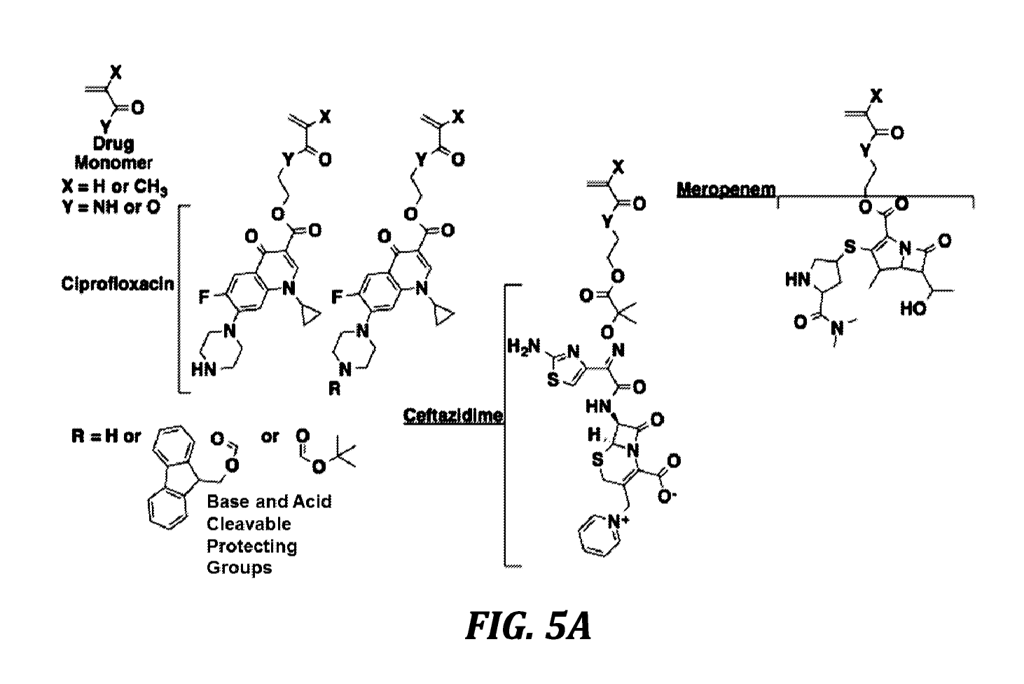

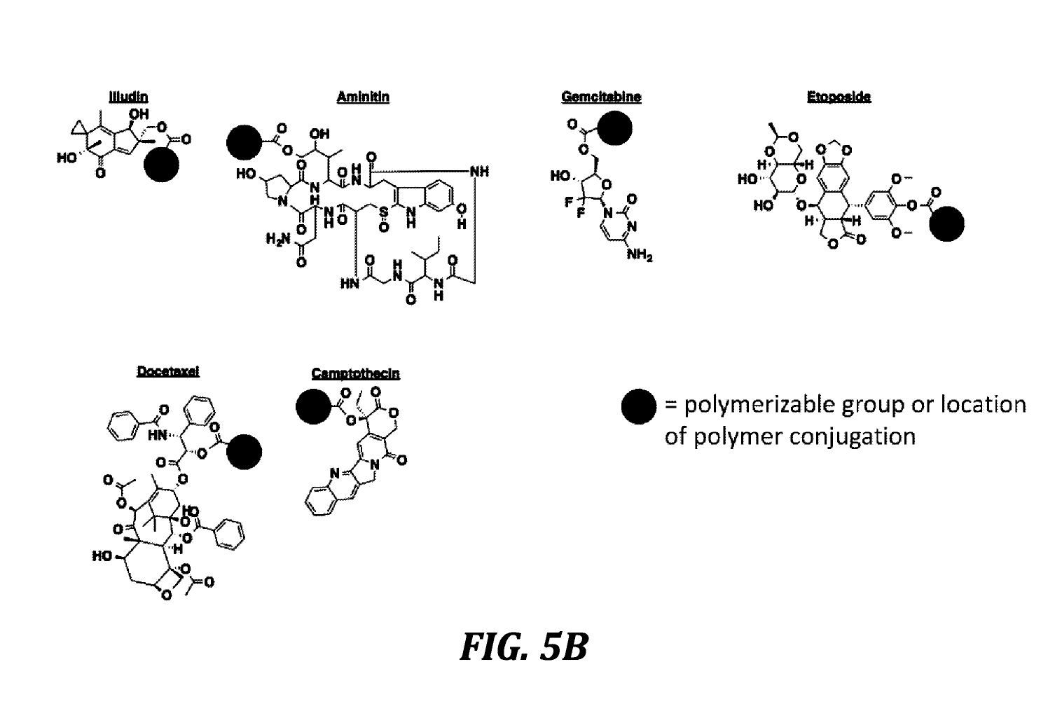

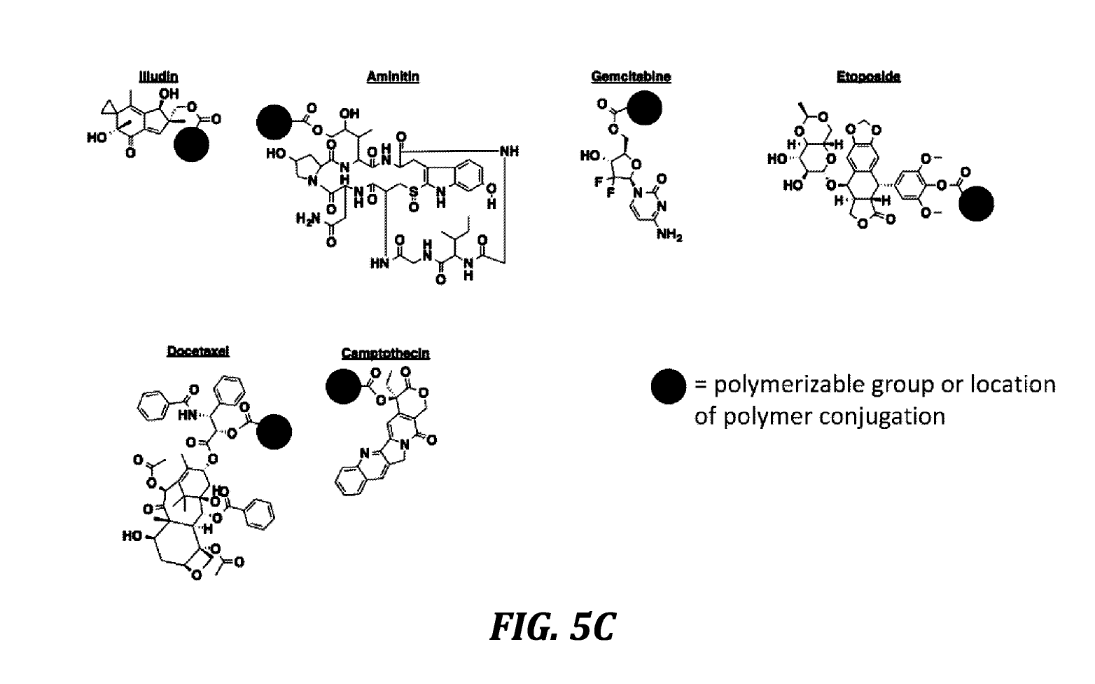

Some examples of therapeutic agents that are conjugated to monomers are provided in FIGS. 5A and 5B. As another example, dasatinib has a hydroxyl group which can be conjugated to carboxylate or anhydride residues to provide an ester, which can be reacted with a monomer (e.g., having an amine moiety) to provide a monomer that can be polymerized with other co-monomers to provide a random copolymer having dasatinib-containing constitutional units.

In some embodiments, ester linkages can be formed with therapeutic agents containing hydroxyl moieties by conjugating the therapeutic agent with a monomer (e.g., PEGMA monomer), or a random copolymer containing a functionalizable constitutional unit (e.g., PEG-containing constitutional unit). As shown in FIG. 5C, post polymerization conjugation can be used for functionalizing a polymer with a therapeutic agent. The therapeutic agent can have more than one functional group (e.g., multiple hydroxyl). Without wishing to be bound by theory, it is believed that an advantage of post-polymerization conjugation is that the steric bulk of the polymer can limit crosslinking between polymer chains. In some instances, post-polymerization conjugation can be relatively easy compared to the synthesis and purification of a therapeutic agent-containing monomer.

Encapsulation of the Therapeutic Agents

In some embodiments, instead of or in addition to conjugating a therapeutic agent to a polymer, the therapeutic agent can be encapsulated (e.g., via non-covalent interactions, such as hydrophobic interactions, hydrogen bonding, and/or ionic interactions) within a random copolymer of the present disclosure. The encapsulated non-covalently bound therapeutic agent can include any of the therapeutic agents listed above. In some embodiments, the encapsulated non-covalently bound therapeutic agent is gentamycin, ciproflaxin, doxycycline, ceftazidime, meropenem, streptomycin, trimethoprim, and/or sulfamethoxazole.

### Advantages of the Particle Assemblies and Polymeric Carriers

Without wishing to be bound by theory, it is believed that covalent bonding of the therapeutic agents to the polymer can enhance drug loading percentage and/or provide a dynamic release mechanism, where the polymer-therapeutic agent linkage is stable in transport but is degraded via chemical and/or enzymatic action upon entering the phagosomal compartment. In the case of particle assemblies, the therapeutic agent is sequestered and protected from the physiological environment until a change in pH triggers the release of the therapeutic agent-containing core random copolymer from the particle-stabilizing shell random copolymer. Furthermore, concurrent pH-triggered activation of the random copolymer can also provide a membrane destabilizing copolymer, thus enhancing the transport of cleaved therapeutic agents into the cytoplasmic compartment.

As described above, the random copolymers and particle assemblies of the present invention can include polyvalent glycan targeting moieties to identify and trigger internalization in target cells, pH-sensing functionalities (e.g., cationic and/or anionic constitutional units) to mediate nanoparticle disassembly and intracellular release of therapeutic agents into the cytoplasmic compartment, and polyethylene glycol (PEG) side chains to impart biocompatibility, transport stability, and to minimize off-target cell uptake and toxicity. By varying the constitutional units, the degradation and disassembly dynamics of the particle assembly and random copolymers can be optimized to enhance bioavailability and provide sustained release of a therapeutic agent over the lifetime of the random copolymers.

Without wishing to be bound by theory, it is believed that glycan side chains on the random copolymers and at the surface of the particle assemblies can direct and concentrate therapeutic agents to cells that express internalizing carbohydrate-binding receptors. For example, ligands that engage multiple carbohydrate recognition domains on macrophage carbohydrate receptor can be potent facilitators of macrophage specific uptake.

In some embodiments, the particle assembly and random copolymers of the present disclosure can provide rapid lung and alveolar macrophage bioavailability from an inhalation route. For example, the particle assembly and random copolymers can be delivered to the deep lung as an aerosol and can deliver therapeutic agents to the cytosol in alveolar macrophage populations. The particle assembly and random copolymers can package the antibiotics into particles with very small sizes to optimize lung transport, distribution and bioavailability. The particle assembly and random copolymers can undergo pH-induced disassembly inside of alveolar macrophage cells to overcome intracellular barriers and depot the drug in the cytoplasm where pathogens (e.g., F. tularensis and/or B. pseudomallei) accumulate. Furthermore, the particle assembly and random copolymers can have polyethylene glycol (PEG) on the outer surface to to impart biocompatibility, transport stability, and to minimize off-target cell uptake and toxicity, even with repeated administrations.

### Conditions

The particle assemblies and random copolymers of the present disclosure can provide broad-spectrum protection against a variety of systemic pathogenic threats. For example, the particle assembly and random copolymers can prophylactically protect against F. tularensis and B. pseudomallei infection, or can treat pre-symptomatic or symptomatic tularemia and melioidosis. In some embodiments, the particle assembly and random copolymer can be used to treat other pathogens (e.g., respiratory pathogens) that have evolved the ability to reside and multiply within lung macrophages, where they are protected from an adaptive immune response and antibiotics, such as plague, anthrax, and glanders. The particle assemblies and random copolymers can mimic these pathogens to create prophylactic and therapeutic depots of therapeutic agents (e.g., antibiotic agents) in lung macrophages, while also providing a direct route for clearance of random copolymers and their biodegradation products.

The following examples are provided to illustrate, not limit, the invention.

Example 1 describes the synthesis and characterization of transferrin-targeted chemotherapeutic delivery systems. Example 2 describes the synthesis of drug-conjugated polymers from drug-containing monomers. Example 3 describes the synthesis and characterization of camptothecin-conjugated random copolymers. Example 4 describes the synthesis and characterization of therapeutic agent-conjugated random copolymers. Example 5 describes a particle assembly formed from binary copolymers. Examples 6 to 11 describe the synthesis, characterization, in vitro evaluation, and in vivo evaluation of antibiotic-loaded particle assemblies.

### EXAMPLES

### Example 1

### Synthesis and Characterization of Transferrin-Targeted Chemotherapeutic Delivery Systems

Reversible addition-fragmentation chain transfer (RAFT) polymerization was employed to prepare a nanoparticulate drug delivery system for chemotherapeutics. The nanoparticles contain a PEG “stealth” corona as well as reactive anhydride functionality designed for conjugating targeting proteins. The multifunctional carrier functionality was achieved by controlling the copolymerization of the hydrophobic monomer lauryl methacrylate (LMA), with a reactive anhydride functional methacrylate (4-Methacryloxyethyl trimellitic anhydride, (TMA)), and a large polyethyleneglycol methacrylate monomer (Mₙ˜950 Da) (O950). RAFT polymerization kinetics of O950 were evaluated as a function of target degrees of polymerization (DP), initial chain transfer agent to initiator ratio ([CTA]ₒ/[I]ₒ), and solvent concentration. Excellent control over the polymerization was observed for target DPs of 25 and 50 at [CTA]ₒ/[I]ₒ ratio of 10 as evidenced by narrow and symmetric molecular weight distributions and the ability to prepare block copolymers. The TMA-functional copolymers were conjugated to the tumor targeting protein Transferrin (Tf). The targeted copolymer was shown to encapsulate docetaxel at concentrations comparable to the commercial single vial formulation of docetaxel (Taxotere). In vitro cytotoxicity studies conducted in HeLa cells show that the Transferrin targeting enhances the cancer killing properties relative to the polymer encapsulated docetaxel formulation.

### Materials

Chemicals and all materials were supplied by Sigma-Aldrich unless otherwise specified. Docetaxel, was obtained from LC Laboratories. 4-Methacryloxyethyl trimellitic anhydride (TMA) was obtained from Polysciences and used as received. Cell mask Far Red, Alexafluor 488 NHS ester, and Hoechst stains were obtained from invitrogen. Spectra/por regenerated cellulose dialysis membranes (6-8 kDa cutoff) where obtained from Fischer. Lauryl methacrylate was passed through a short column of basic alumina. PD10 columns were obtained from GE life sciences. MTS cytotoxicity kits were obtained Promega. HeLa cells, human cervical carcinoma cells (ATTC), were maintained in minimum essential media (MEM) containing L-glutamine (Gibco), 1 percent penicillin-streptomycin (Gibco), and 10 percent fetal bovine serum (FBS, Invitrogen) at 37° C. and 5 percent CO₂. O950 (Aldrich) (30 g) was dissolved in 70 g of tetrahydrofuran (THF) and then passed through a 6-inch plug activated basic alumina. The monomer was collected slowly due to height of packed Al₂O₃ and the viscosity of the concentrated monomer solution. After collection, the THF was removed under reduced pressure using a rotary evaporator followed by a high vacuum line. The PEGMA 950 solution, which became a waxy solid under reduced pressure, was gently melted by immersion of the flask in a warm water bath under high vacuum. NMR analysis confirmed the presence of 3 percent (by mass) residual THF which was accounted for in subsequent polymerization calculations.

The RAFT copolymerization of LMA, TMA, and PEGMA 950 was conducted in inhibitor free dioxane at an initial total comonomer concentration of 10 wt. percent with an CTA to initiator ratio)([CTA]ₒ/([I]ₒ) of 5 to 1 with an monomer to CTA ratio ([M]ₒ/[CTA]ₒ) of 50 to 1. Polymerization were purged with nitrogen for 1 hour and then heated at 70° C. for 24 hours. After this time, the polymerization solutions were transferred to spectrapor regenerated cellulose dialysis membranes (preequilibrated in deionized water) and then dialyzed against acetone at 5° C. for 1 week. Following dialysis, the polymer solution in acetone was filtered through a plug of cotton after which time the acetone was removed via rotary evaporation. The resultant viscous solid was then dried under high vacuum for 48 hours. A representative procedure is as follows: To a 50 mL round bottom flask was added O950 (3.12 g, 3.28 mmol), LMA (2.50 g, 9.83 mmol), TMA (1.00 g, 3.28 mmol), CTP (92 mg, 0.033 mmol), azobbiscyanovaleric acid (ABCVA) (18.4 mg, 0.066 mmol), and Dioxane (27 g). The polymerization solution was then purged with nitrogen for 60 minutes and then transferred to a preheated oil bath at 70° C. and allowed to react for 24 hours. Copolymer composition was determined by integration of the aromatic resonances (7.70-8.80 ppm) and the methoxy resonance (3.37 ppm) associated with 3 protons from TMA and O950 respectively allowed algebraic determination of the LMA content via subtraction of the corresponding methylene and methyl resonance for each of these comonomers from the backbone region (0.5-3.0 ppm).

Conjugation of Transferrin to poly[(LMA_{co}TMA_{co}O950)

Transferrin was conjugated to the random copolymer via reaction of pendant anhydride residues incorporated throughout the copolymer via the methacrylate comonomer (TMA) with lysine residues on the proteins. The copolymer and protein stocks were prepared at 75 and 21 mg/mL in ethanol and buffer respectively. The protein solution was diluted with additional buffer such that the final protein concentration following addition of the ethanolic polymer stock was 0.3 mg/mL. To this solution the copolymer in ethanol was added to produce the desired polymer/protein molar ratios over a range of 1:1 to 128:1. The degree of protein conjugation to the polymer was verified using polyacrylamide gel electrophoresis (PAGE) using Mini-PROTEAN TGC precast gels (4-20 percent) (BIORAD) with tris-glycine-SDS (10×stock=0.25 M tris, 1.92 glycine, 1 percent SDS) (national diagnostics). The gel was run for 1 hour at 150 Volts at room temperature and subsequently stained for 18 hours in GelCode blue and destained overnight with deionized water.

Doxetaxel was codissolved with the poly(LMA_{co}TMA_{co}PEGMA 950) according to the formulation parameters outlined in Table 1. The concentrated docetaxel-ethanol-polymer was then diluted with 5 percent dextrose solution containing 0.2 M HEPES buffered to pH 7.4 to a final drug concentration of 0.5 mg/mL. The solutions where then sterile filtered through a 0.2 μm filter and then stored at 5° C. For in vitro toxicity experiments, where the desired drug-loading is significantly lower than the corresponding clinical formulations, the polymer concentration was fixed at 0.2 mg/mL. To a solution of copolymer (400 μg) in dextrose HEPES buffer 20 mM pH 7.4 (93 μL) was added 5 μL of docetaxel as stock solutions in ethanol. This solution was then incubated at 5° C. overnight at which time 1900 μL of media was added. The solution was then sterile filtered with a 0.2 μm filter and 200 μL was added to 96 well plates such that the final DTX concentration was between 100 and 0.5 nM.

TABLE 1
Theoretical and experimentally determined copolymer composition,
number average molecular weights (Mn), and molar mass distributions (D)
for a series of PEGMA 950, LMA, and TMA copolymers prepared by RAFT.

| | % | % | % | % | % | % | | | | D_{H} | D_{H} |
|---|---|---|---|---|---|---|---|---|---|---|---|
| | TMA | 950 | LMA | TMA^{a} | 950 | LMA^{a} | Mₙ^{b} | | CMC | Poly | DTX |
| Poly. # | (Feed)\ | (Feed) | (Feed) | (Exp.) | (Exp.) | (Exp.) | (Da) | [symbol] ^{b} | (μg/mL) | (nm)^{a} | (nm)^{a} |
| B-1 | 25 | 15 | 60 | 21 | 11 | 68 | 26600 | 1.10 | 26 | 6.2 | 4.9 |
| B-2 | 25 | 20 | 55 | 23 | 14 | 63 | 31600 | 1.12 | 34 | 6.7 | 4.3 |
| B-3 | 25 | 25 | 50 | 23 | 18 | 59 | 37800 | 1.09 | 52 | 5.1 | 5.6 |

^{a}As determined by 500 mhz 1H NMR spectroscopy in CDCl3 by evaluation of PEGMA 950 Methoxy resonance [A] at 3.39 ppm, the TMA aromatic resonances [B] between 8 and 9 ppm, and the combined ester region [C] at 4.1 and 5.0 ppm using the formulas: 1H PEGMA 950 = [A]/3, 1H TMA = [B]/3, and 1H LMA = ([C] − 2/3 × [A] − 2/3 × [B])/2.
^{b}As determined by size exclusion chromatography using Tosoh SEC TSK-GEL α-3000 and α-4000 columns (Tosoh Bioscience, Montgomeryville, PA) connected in series to an Agilent 1200 Series Liquid Chromatography System (Santa Clara, CA) and Wyatt Technology miniDAWN TREOS, 3 angle MALS light scattering instrument and Optilab TrEX, refractive index detector (Santa Barbara, CA). HPLC-grade DMF containing 0.1 wt. % LiBr at 60° C. was used as the mobile phase at a flow rate of 1 ml/min.

The cytotoxicity of drug-loaded micelles as well as the drug free copolymer where evaluated in HeLa cells using the CellTiter 96 Aqueous One Solution Cell Proliferation Assay (MTS) (Promega Corp., Madison, Wis.). HeLa cells were seeded at a density of 5000 cells/well in 96-well plates and allowed to adhere for 24 hours. The media was then replaced with 200 μL of fresh media containing the unloaded and drug loaded micelles at the appropriate concentrations. After 72 hours the media was replaced with new media and the cells were evaluated using the CellTiter MTS assay according to the manufactures instructions. The absorbance at 490 nm was evaluated using Tecan Safire 2 microplate reader. Untreated cells in media were used as a negative control. All experiments were carried out at a series of four replicates for at least two experiments.

Absolute molecular weights and dispersity indices D were determined using Tosoh SEC TSK-GEL α-3000 and α-4000 columns (Tosoh Bioscience, Montgomeryville, Pa.) connected in series to an Agilent 1200 Series Liquid Chromatography System (Santa Clara, Calif.) and Wyatt Technology miniDAWN TREOS, 3 angle MALS light scattering instrument and Optilab rEX, refractive index detector (Santa Barbara, Calif.). HPLC-grade DMF containing 0.1 wt. percent LiBr at 60° C. was used as the mobile phase at a flow rate of 1 ml/min.

The critical micelle concentration (CMC) for the copolymer micelles were determined using Rhodamine 6G as a fluorescence probe. The concentration of copolymer was varied between 1 and 1000 μg/mL, with a fixed concentration of Rhodamine 6G of 10 μM. The fluorescence spectra were recorded using a Tecan Safire 2 microplate reader with an excitation and emission wavelength of 480 and 550 nm, respectively. The CMC was estimated as the cross-point when extrapolating the intensity at 550 nm between low and high concentration regions.

The intracellular distribution of fluorescently labeled transferrin-polymer conjugates was evaluated in cells using live-cell fluorescence microscopy. Cells were seeded in chamber slides at a concentration of 5000 cells/well. After allowing the cells adhere for 18 hours the media was replaced with fresh media containing the appropriate concentration of the copolymer-transferrin conjugates. Thirty minutes prior to imaging Hoechst (5 μg/mL) was added to the media and allowed to incubate at 37° C. After this time, the media was carefully aspirated and replaced with PBS. Immediately prior to imaging individual wells were aspirated and then treated with 2 μL of a 10 mg/mL cell mask stock in 200 μL of ice cold PBS. After 3 minutes the PBS was removed and washed three times with additional ice cold PBS. The mediChamber slides were placed on a Live-Cell Fluorescence Microscope (Nikon Ti-E) equipped with an environmental control chamber. Cells were imaged with a mercury lamp and a 100× objective using the following filter sets: 480/40, (EX) and 535/50 nm (EM) for AF488, Hoechst and Cell Mask Deep red. (Chroma 49000 Series, Rockingham, Vt.). For each image stack, 24 z-sections with a 0.5 um step size were collected using a ⅛ neutral density filter and 400 millisecond exposure. After image acquisition, image stacks were deconvolved using object-based measurement software, Velocity (Perkin Elmer), to remove out-of focus fluorescence for the identification of conjugate containing compartments. To deconvolved image stacks, point spread functions were calculated for the green, blue, and deep red channel and applied using 25 iterations to reach a near 100 percent confidence interval.

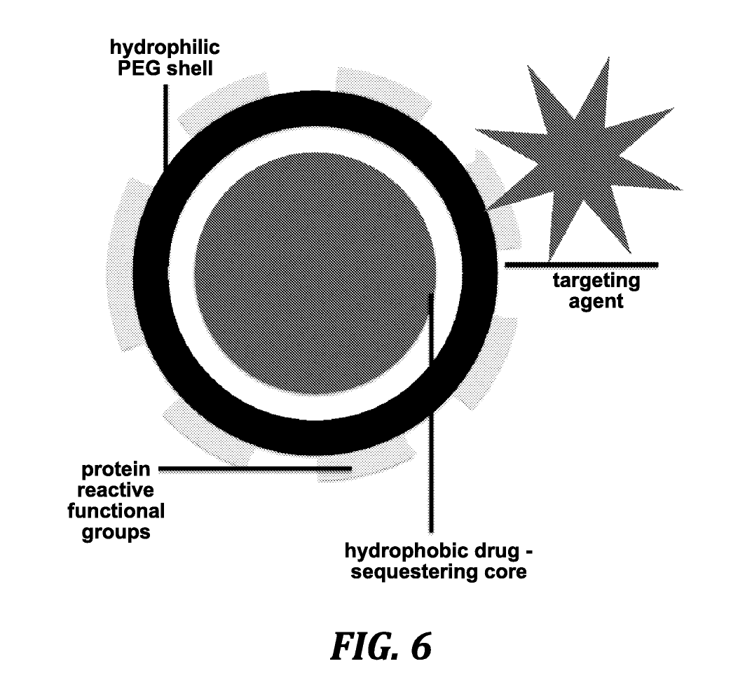

Shown in Scheme 2 is the synthetic strategy for the preparation of polyethylene glycol (PEG) containing copolymers capable of efficiently encapsulating hydrophobic drugs such as the antineoplastic agent docetaxel. Under aqueous conditions these materials are designed to self-assemble to form micelles with phase separated LMA residues stabilized in solution by hydrophilic O950 residues. This combination of monomers closely mimics the physiochemical properties of the clinically established surfactant-based drug stabilizer Polysorbate 80 (PS80). PS80 stabilized formulations of docetaxel show high intrinsic tumor killing activity but are associated with severe dose-limiting toxicity. In order to minimize these toxic side effects and enhance the effectiveness of current taxane-based therapies, the drug delivery system outlined in Scheme 2 is designed employ tumor specific targeting ligands. These ligands are conjugated to the hydrophilic micelle corona via the reaction of amine residues (present on many large proteins and antibodies) with polymeric anhydride residues. These conjugation reactions can be conducted directly in physiological saline without the need for protein modification reactions (Scheme 2). Anhydride functionality is easily integrated directly into the copolymers through the addition of 4-Methacryloxyethyl trimellitic anhydride (TMA) to the polymerization. A schematic representation of the copolymer drug delivery system integrating tumor specific transferrin targeting groups, a hydrophilic polyethylene glycol (PEG) stealth corona, and a hydrophobic drug sequestering core is shown in FIG. 6.

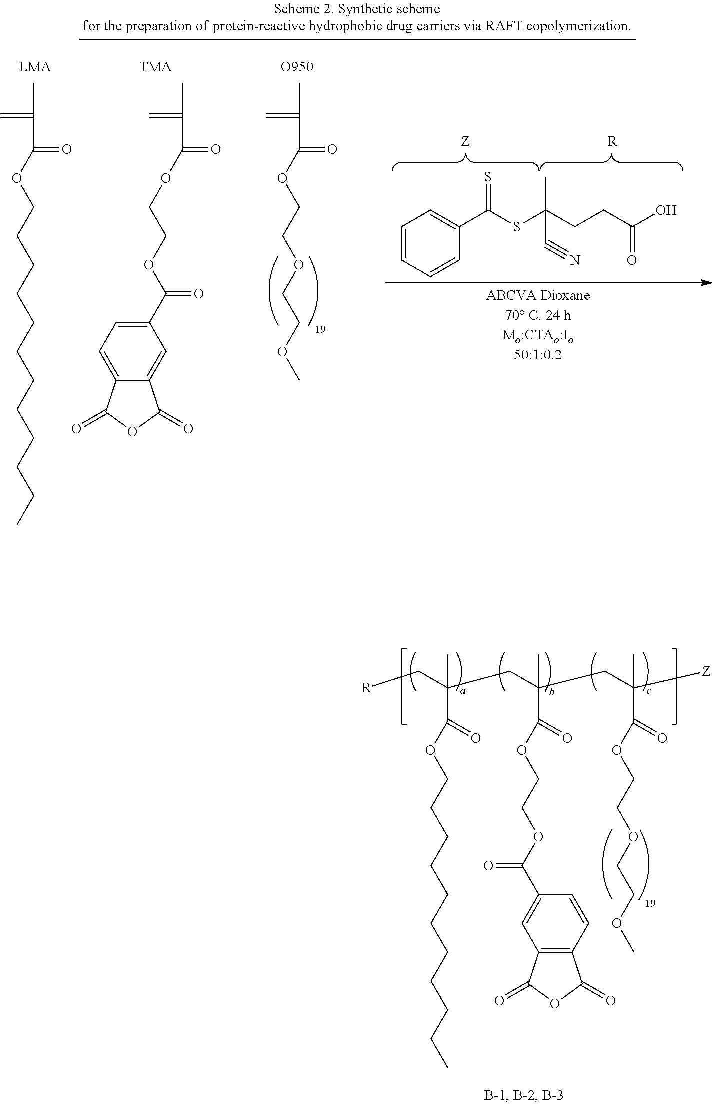

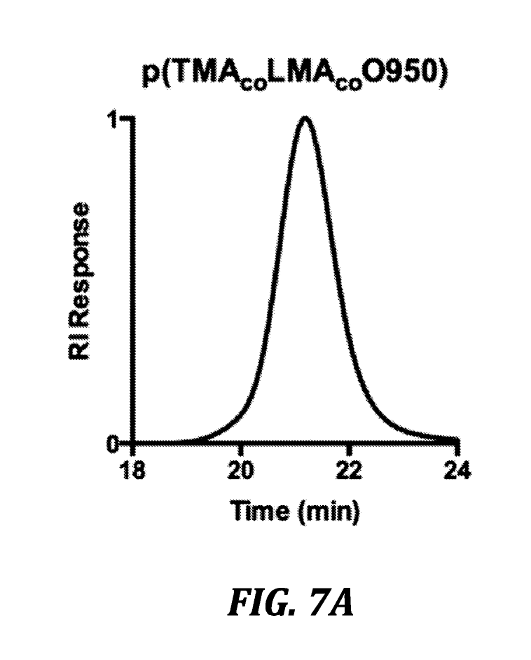

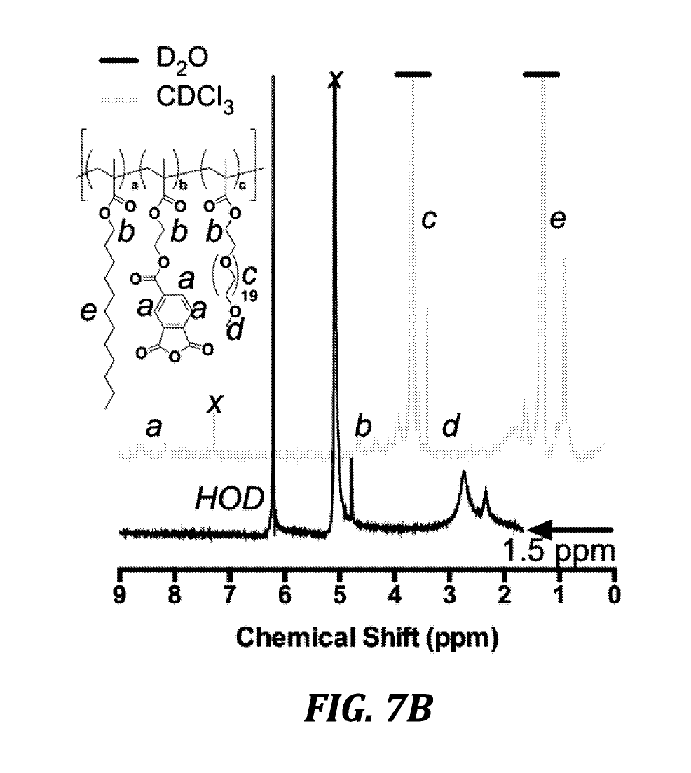

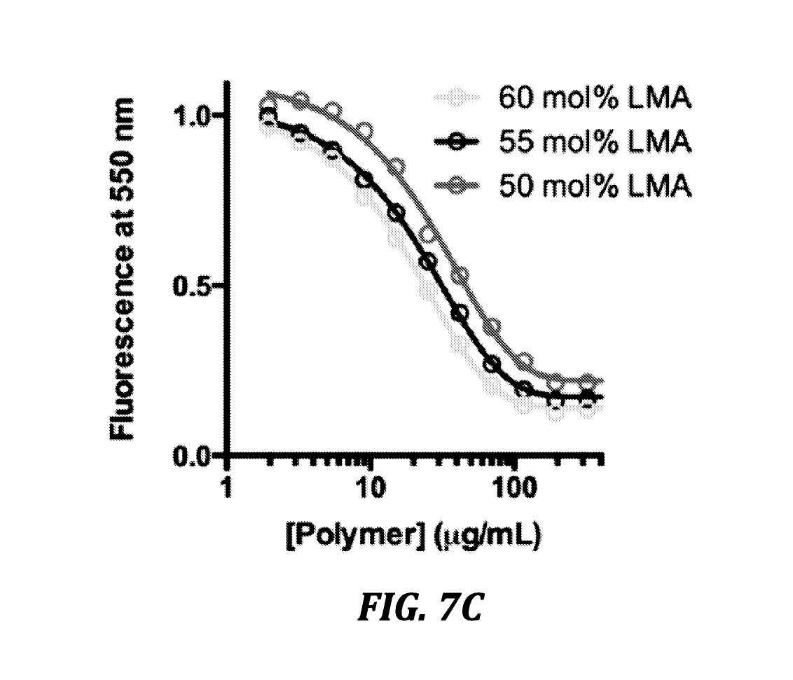

A series of protein-reactive drug nanocarriers (B-1, B-2, B-3) was prepared, targeting a range of hydrophobic LMA to hydrophilic O950 ratios at a fixed molar feed ratio of 25 percent for the reactive TMA comonomer as outlined in Scheme 2. The molecular weight, D, and composition data for these copolymers are outlined in Table 1. Shown in FIGS. 7A and 7B-7C are the molecular weight distribution and ¹H NMR spectrum for polymer B-3, which was conducted with an initial molar percentage of TMA:O950:LMA of 25:25:50 percent. The molecular weight distribution for the copolymerization is quite narrow and symmetric and is representative of the other copolymer compositions outlined in Table 1. The ¹H NMR spectrum for polymer B-3 conducted in CDCl₃, which is a good solvent for all three comonomer residues in the copolymer, show resonances associated with each of the individual comonomer units. For example, the large aliphatic resonance associated with the LMA sidechain (v) is visualized as an intense resonance at 1.28 ppm. Also present are the intense resonances associated with the O950 ethylene oxide repeat (iii) units and —OCH3 (iv) at 3.66 and 3.39 ppm as well as the aromatic TMA resonances (i) between 8 and 9 ppm. Dissolution of this copolymer in D₂O shows a strong attenuation of the hydrophobic resonances while the O950 resonances remain prominent. This result is consistent with the formation of the desired micellar morphology with the hydrophobic PEGMA 950 residues stabilizing the hydrophobic LMA residues in aqueous solution. Dynamic light scattering results suggest that these micelles are small with sizes around 5 nM which is consistent with intramolecular or unimolecular micelles.

The critical micelle concentration for the copolymers was evaluated as a function of hydrophobic lauryl methacrylate content for copolymers containing between 59 and 77 mol percent LMA over a concentration range of 1 and 1000 μg/mL in phosphate buffer (pH=7.4) using rhodamine 6G as a polarity sensitive fluorophore (FIG. 7B). Rhodamine 6G is strongly fluorescent in water but upon localization in a less polar environment this fluorescence is usually quenched. While all three copolymers showed similar fluorescence trends higher LMA containing copolymers showing slightly lower CMCs (Table 1). These values were found to be between 52 μg/mL for copolymer B-3 containing 59 percent LMA and 26 μg/mL for copolymer B-1 containing 68 percent LMA. Copolymers containing higher mole fractions of LMA (e.g., n_{f} LMA=80 percent) were not readily dispersible in aqueous solution but could be dispersed in water to form 60 nm particles following the formation of films cast from chloroform (not shown).

Conjugation of the copolymer to large proteins was investigated using Transferrin under a variety of pH and analyte conditions. Transferrin receptors are highly overexpressed in a number of cancers including cervical cancers. Protein conjugation studies were conducted by first dissolving the polymer in ethanol at a concentration of between 50-500 mg/mL. The concentrated ethanolic stock was then induced to form micelles by dilution directly into the appropriate buffer. These conditions were designed to closely match formulation of docetaxel (Taxotere) which is consists of a concentrated stock of the drug plus ethanol in polysorbate 80 and a second aqueous diluent. The effect of pH on the conjugation of polymer B-1 to Transferrin was evaluated as a function of solution pH.

Evaluation of Transferrin conjugation via gel electrophoresis following overnight incubation with poly(LMA_{co}O950_{co}TMA) was conducted, where polyacrylamide gel electrophoresis for transferrin and Trastuzumab monoclonal antibody conjugation reactions were conducted at various polymer to protein ratios. The degree of protein conjugation to the polymer was verified using polyacrylamide gel electrophoresis (PAGE) using Mini-PROTEAN TGC precast gels (4-20 percent) (BIORAD) with tris-glycine-SDS (10×stock=0.25 M tris, 1.92 glycine, 1 percent SDS) (national diagnostics). The gel was run for 1 hour at 150 Volts at room temperature and subsequently stained for 18 hours in GelCode blue and destained overnight with deionized water. In the gel electrophoresis, the free Transferrin band remains approximately constant at both pH 9.6 and 8.3 with only slight attenuation at the highest polymer to protein ratio. In striking contrast the conjugation reactions conducted in pH 7.4 HEPES buffer containing 5 percent glucose show significant reductions in the free Transferrin band at even the lowest polymer to protein ratios with complete conjugation at a ratio of approximately four. This significant difference in conjugation efficiency could either be related to the solution pH or the presence of 5 wt percent glucose in the buffer. Given the significant differences in conjugation efficiency and the clinical relevance of physiologically buffered glucose solutions these conditions were employed in all subsequent conjugation reactions.

The ability of the protein-reactive copolymers to encapsulate docetaxel under clinically relevant conditions was evaluated using formulation conditions similar to the single vial formulation of Taxotere 20 mg vials. This formulation consists of 20 mg of Taxotere in 1 mL of 50:50 (v/v) polysorbate 80 to ethanol which is then diluted into an IV of 5 percent dextrose or 0.9 percent NaCl to yield a final docetaxel concentration of between 0.3 and 0.74 mg/mL. This solution is reported to be stable for 4 hours if stored at ambient temperature and lighting conditions. Based on these formulation parameters, a range of polymer to docetaxel ratios between 5 and 11 percent (w/w) were evaluated as a preconcentrate in 50 percent ethanol to polymer (v/v). Following complete dissolution of the docetaxel in the polymer ethanol mixture, the resultant preconcentrate was then diluted with 5 percent dextrose containing 20 mM HEPES buffer pH 7.4 to a final docetaxel concentration of 0.5 mg/mL. These buffer conditions were selected because they are already commonly employed in clinical IV formulations and were shown in the previous section to provide excellent protein conjugation to the reactive polymer scaffold. Under all formulation conditions evaluated, the preconcentrate stock completely dissolved in the aqueous buffer without any observable precipitate. Dynamic light scattering of the resultant aqueous solutions showed that the particles approximately 5 nm and remained stable for extended periods of time without any evidence of precipitation. Indeed, subsequent dynamic light scattering measurements conducted after 14 days in solution at 5° C. showed that particle sizes remain approximately constant with no observable precipitation.

Live cell imaging experiments conducted with fluorescently (ALEXA 488) labeled Transferrin confirm that the polymer bound protein retains the ability to access intracellular compartments via receptor-mediated endocytosis. Fluorescence from Transferrin was strongly localized within endosomes/lysosomes upon incubation with HeLa cells for 1 hour at 37° C. Hoechst and CellMask were also employed in order to visualize the nucleus and cell membrane respectively.

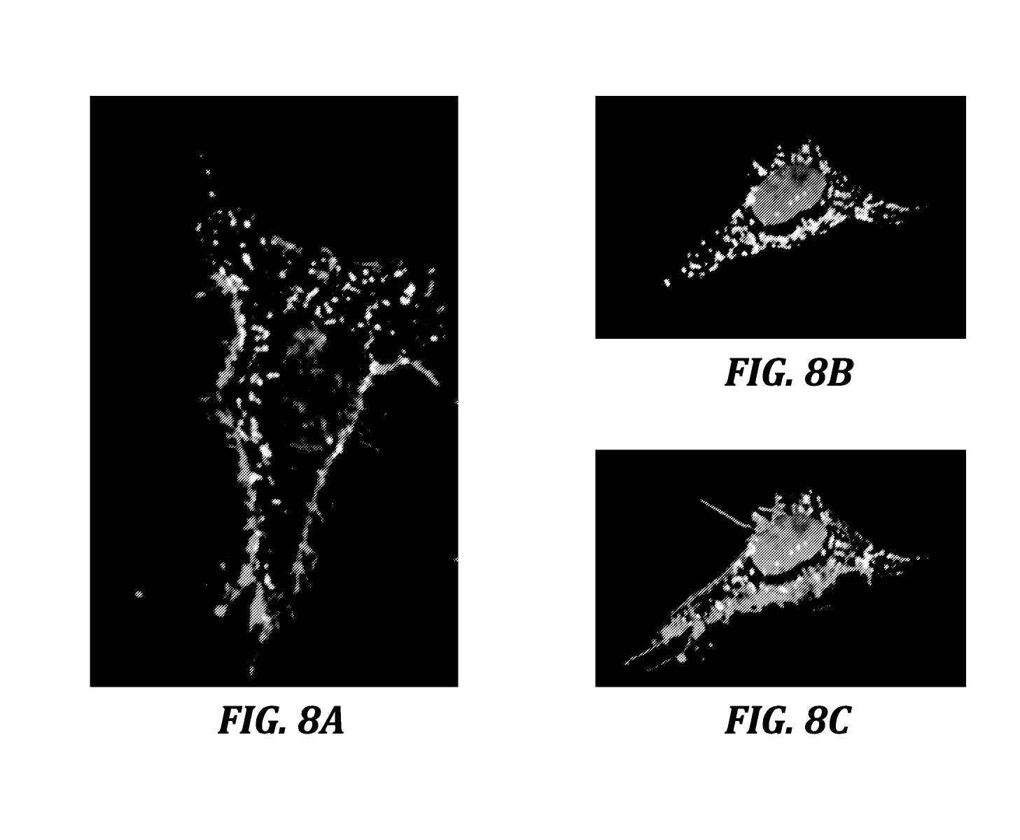

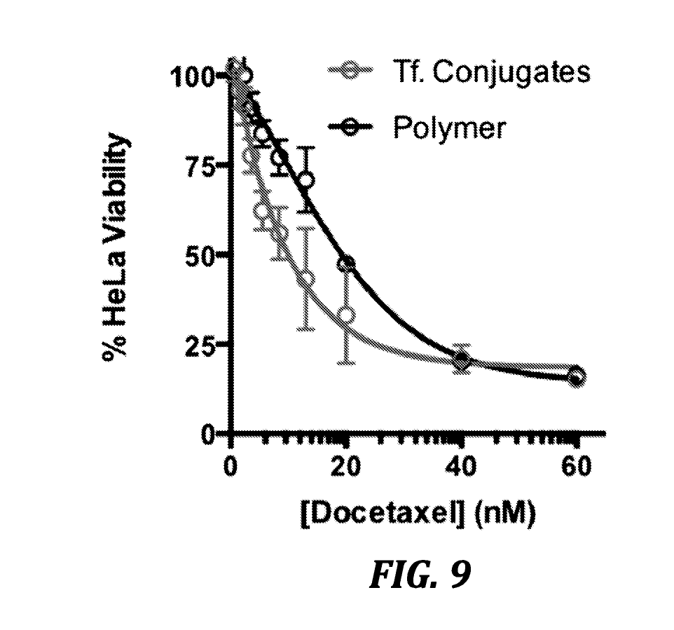

The cytotoxicity of the copolymer encapsulated docetaxel was evaluated in HeLa (human cervical cancer) cell lines that overexpress Transferrin receptors. Formulations were prepared over a range of docetaxel concentrations between 1 and 100 nM. Shown in FIGS. 8A to 8C and 9 are the results of HeLa cell viability experiments in which transferrin, which was employed as the targeting ligand, shows improvements in the dose-dependent toxicity. Transferrin receptors continually cycle from the cell surface to intracellular compartments with an average period of 7 minutes required to turn over the total population of surface receptors. Once internalized these receptors have an intracellular resonance time of approximately 21 minutes suggesting that approximately three-quarters of the transferrin receptor are present inside the cells at any moment. Evaluation of the 1d50 values in HeLa cells following a 72 hour incubation with both the polymer encapsulated docetaxel and the corresponding Transferrin conjugates showed values of 19 and 9.7 nM respectively. These studies demonstrated the greater and sustained antiproliferative activity of the transferrin targeted nanoparticles in dose- and time dependent studies in MCF-7 and MCF-7/Adr cells. The greater antiproliferative properties of the Tf-targeted system is attributed to higher levels cellular uptake and reduced exocytosis of the targeted nanoparticles.

Thus, the random copolymer copolymers were shown to efficiently encapsulate the potent chemotherapeutic agent docetaxel with formulation parameters comparable to the clinically established Taxotere. Aqueous formulations of the copolymer were shown to spontaneously form covalent bonds with Transferrin under clinically relevant buffer conditions. The presence of Tf targeting was shown significant improvements in docetaxel for the polymer-encapsulated docetaxel are comparable to the parent compound while fluorescent microscopy show substantial levels of internalized Tf-protein conjugates. These results taken together suggest that the targeted drug delivery technology outlined in this report could enhance the therapeutic index of clinically well-established chemotherapeutic agents by directing their accumulation in tumor tissue.

### Example 2

### Drug-Conjugated Polymers from Drug-Containing Monomers

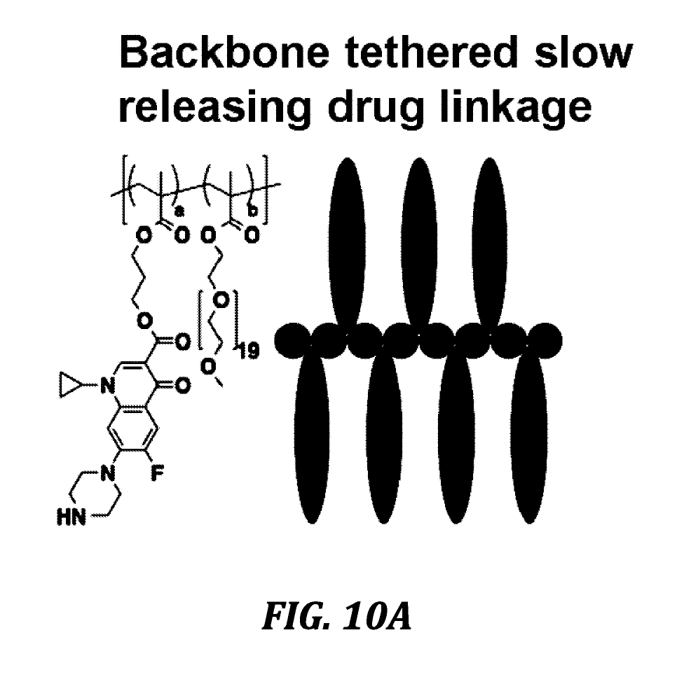

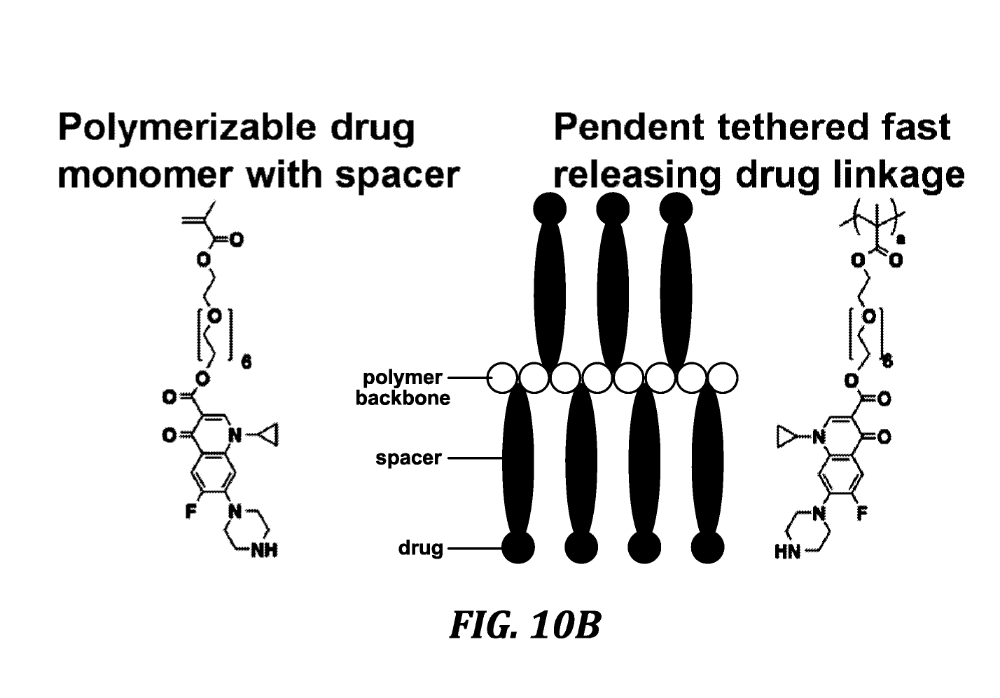

Therapeutic Molecules can be introduced into polymers via either conjugation or through the use of a polymerizable drug monomer. As shown in FIG. 10A, for ester-linked drug molecules where the linkage is short, slow sustained drug release was observed. Faster release can be observed for constructs where the drug is attached to terminal hydroxyl groups that are present on some commercially available PEGMA monomers. FIG. 10B shows a short PEGMA (ovals) containing a terminal hydroxyl group was conjugated to boc-protected ciprofloxacin and then copolymerized to form a polymer where the drug (filled circles) is away from the low dielectric (slow hydrolyzing) polymer backbone (empty circles). The combination of slow and fast releasing drug linkages can provide advantages for many therapeutic applications.

### Example 3

### Synthesis and Characterization of Camptothecin-Conjugated Random Copolymer

### Synthesis of mono-2-(methacryloyloxy)ethyl succinate camptothecin ester (CamSMA)

To a 100 mL round bottom flask was added camptothecin (0.747 g, mmol), mono-2-(Methacryloyloxy)ethyl succinate (SMA) (2.47-g, 10.73 mmol), 4-dimethylaminopyridine (DMAP) (1.31 g, 10.73 mmol), and 74.7 mL anhydrous dimethyl sulfoxide (DMSO). After the reagents were dissolved with light sonication, N,N′-dicyclohexylcarbodiimide (DCC) (2.21 g, 10.73 mmol) was added. The solution was then allowed to react in the dark for 24 hours. After this time the solution was filtered through a plug of cotton and then precipitated in 1000 mL of 150 mM HEPES buffer pH 8.4 that had been precooled to 5° C. The precipitated was then filtered, washed with deionized water, and then dissolved in warm acetone. This solution was then filtered through a plug of cotton and then precipitated in petroleum ether. The precipitate was then dried under high vacuum for 24 hours.

### RAFT Copolymerization of CamSMA and Polyethyleneglycol Methacrylate 950 Da (O950)

To a round bottom flask was added CamSMA (0.252 g, 0.000449 mol), O950 (0.854 g, 0.000899 mmol), 4-Cyano-4-(phenylcarbonothioylthio)pentanoic acid (CTP) (15.7 mg, 0.0539 mmol), azobiscyanovaleric acid (ABCVA) (3.02 mg, 0.0107 mmol), and dmso (4.42 mL). The solution was then septa sealed, purged for 30 minutes with nitrogen and then allowed to polymerized at 70° C. for 18 h.

After this time the camptothecin-conjugated random polymer (C) was precipitated into a large excess of diethyl ether and then dried overnight under high vacuum. The polymer was then further purified by dissolving it in deionized water and then filtering it through a PD10 desalting column according to the manufactures instructions.

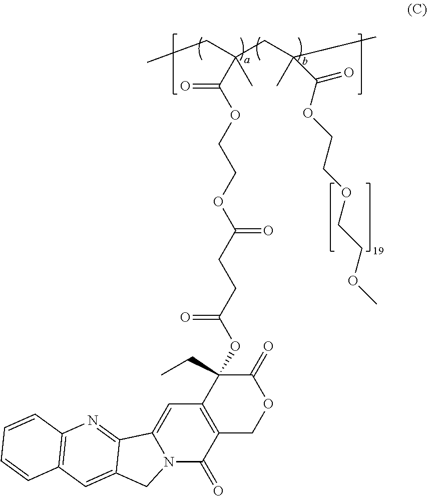

### Example 4

### Synthesis and Characterization of Therapeutic Agent-Conjugatable Random Copolymers

To a round bottom flask was added mono-2-(methacryloyloxy)ethyl succinate tertiary butyl (boc) carbazate (bocSMA) (1.0 g, 2.9 mmol), N,N-diethylaminoethyl methacrylate (DEAEMA) (3.22 g, 17.4 mmol), butyl methacrylate (BMA) (1.65 g, 11.6 mmol), CTP (81 mg, 0.290 mmol), ABCVA (16.3 mg, 0.058 mmol), and dioxane (13.7 mL). The polymerization solution was then septa-sealed and then purged with nitrogen for 30 minutes. After this time the polymerization solution was added to a preheated water bath at 70° C. and allowed to react for 18 hours. After this time the polymerization solution was dialyzed against acetone for 3 days and then the therapeutic agent-conjugatable polymer was isolated via rotary evaporation. A therapeutic agent (e.g., doxycycline, ciprofloxacin, meropenem, etc.) can then be conjugated to the polymer, following removal of the BOC protecting group to expose the pendant reactive hydrazone moiety.

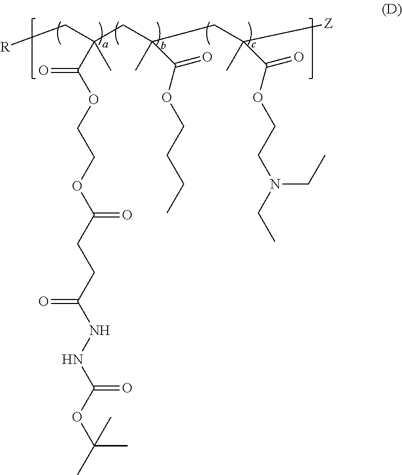

### Example 5

### Particle Assembly from Ionic Polymers

### RAFT Copolymerization of bocSMA and Polyethyleneglycol Methacrylate 300 Da (O300)

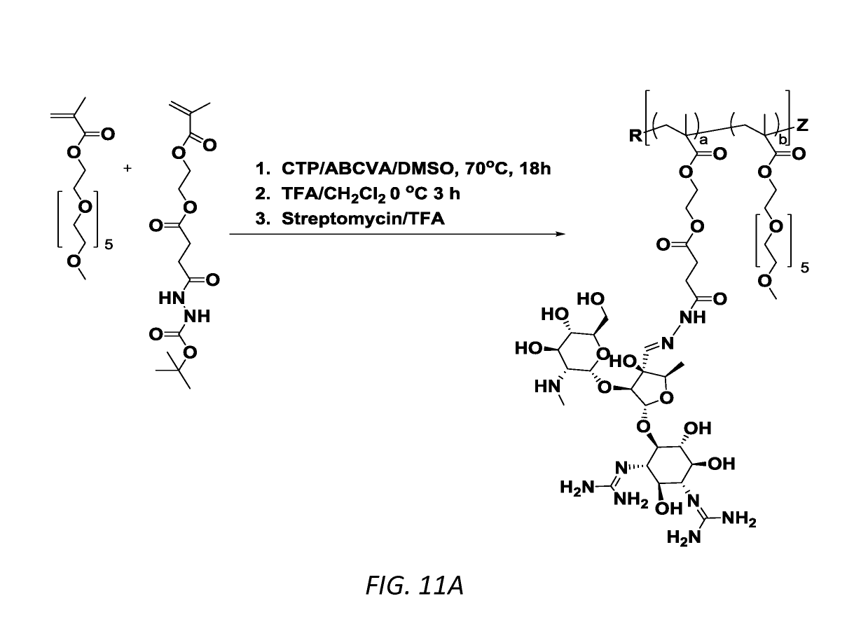

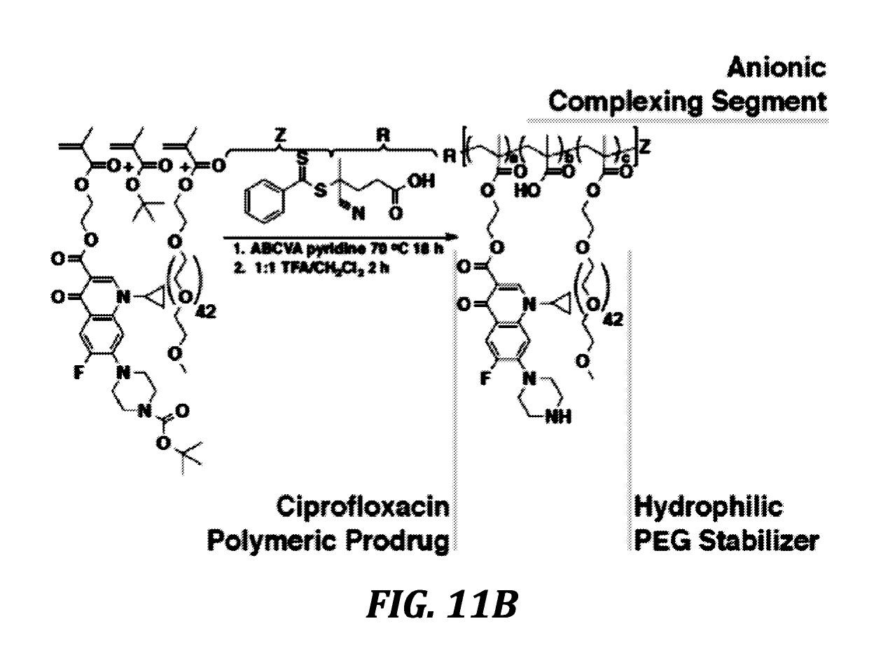

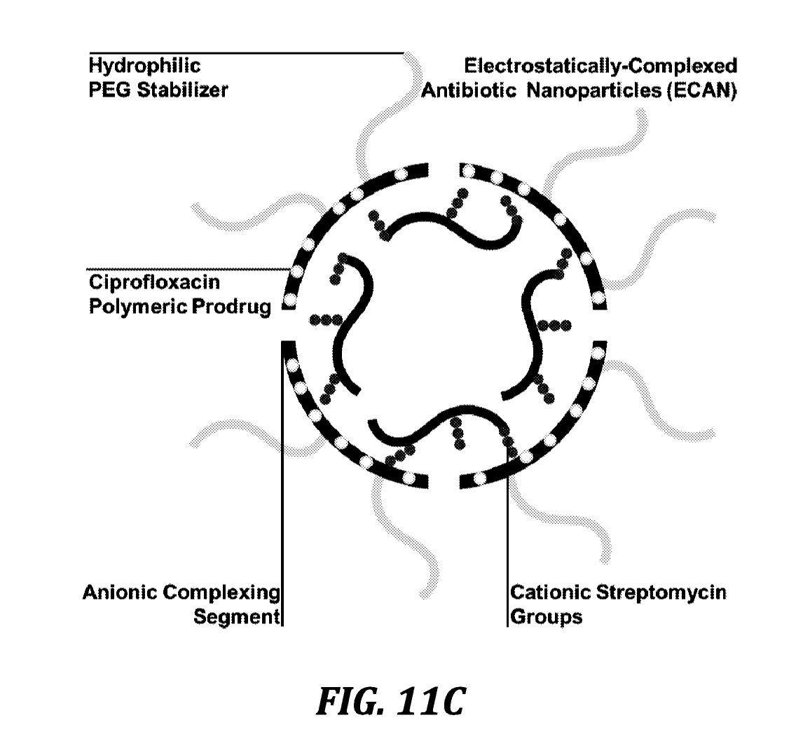

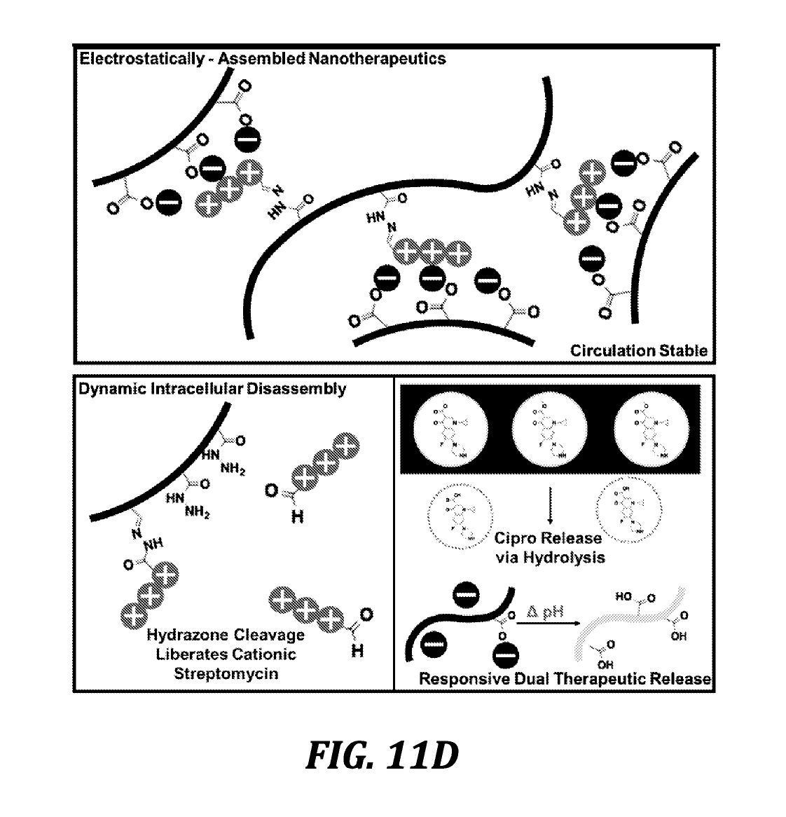

To a round bottom flask was added PEG methacrylate monomethyl ether (FW 300 Da) (2.0 g, 5.8 mol), bocSMA (1.74 g, 5.8 mmol), 4-Cyano-4-(phenylcarbonothioylthio)pentanoic acid (CTP) (65 mg, 232 mmol), azobiscyanovaleric acid (ABCVA) (6.5 mg, 23 mmol), and DMSO (15.0 mL). The solution was then septa sealed, purged for 30 minutes with nitrogen and then allowed to polymerized at 70° C. for 18 h. After this time the random polymer was precipitated into a large excess of diethyl ether and then dried overnight under high vacuum. The tertiary butyl boc protecting groups were subsequently removed by incubating the polymer in trifluoroacetic acid (TFA) (10 mL/TFA per 5 polymer) for 3 hours. The polymer was then isolated by precipitation into a large excess of diethyl ether. Conjugation of streptomycin was achieved by dissolved the hydrazone functional polymer and streptomycin in a saturated streptomycin/water solution with the addition of a catalytic amount of TFA. A ciprofloxacin random copolymer was synthesized in an analogous manner. FIGS. 11A and 11B show the synthetic scheme of a cationic streptomycin-conjugated random copolymer and a ciprofloxacin conjugated anionic random copolymer.

Referring to FIG. 11C, when assembled, the two polymers form a nanoparticle assembly consisting of an outer ciprofloxacin containing random copolymer and an inner streptomycin containing random copolymer. The outer ciprofloxacin-containing random copolymer contains constitutional units that facilitate nanoparticle solubility (e.g., PEG and anionic moieties), and biocompatibility/low toxicity (e.g., PEG moieties). The inner streptomycin-containing random copolymer can form ionic bonds with the outer random copolymer.

Referring to FIG. 11D, streptomycin is linked to the inner random copolymer via an acid cleavable hydrazone bond. Upon internalization into target cells/tissue the low pH environment cleaves this bond releasing the cationic streptomycin residues. Concurrent protonation of the carboxylic acid residues in the outer core further disrupts nanoparticle stability facilitating disassembly. As shown in FIG. 11D, the outer shell contains covalent ester linked ciproflaxin that provides simultaneous delivery of multiple therapeutic agents.

### Example 6

### Antibiotic-Loaded Particle Assembly Synthesis and Characterization

Meropenem is recommended for treatment of B. pseudomallei infection but penetrate mammalian cells poorly. It is believed that intracellular delivery of the drug will enhance killing of intracellular bacteria such as B. pseudomallei. Meropenem-containing monomers are synthesized via esterification of carboxylic acid present on the parent drug with 2-hydroxy ethyl methacrylate. A tert-butyloxycarbonyl (BOC) protecting group is used to prevent reaction of the secondary amine functionality of meropenem. The resultant antibiotic prodrug monomer contains a methacrylate functionality that will efficiently copolymerize with the other methacrylate-based co-monomers. Ester-linked drugs provide a good stability balance in that they are sufficiently stable to allow for reasonable in vivo circulation times while providing sufficiently fast cleavages rates under low pH conditions in the presence of endosomal lysosomal enzymes. This linkage strategy has been applied successfully both in vitro and in vivo to delivery chemotherapeutic agents, as described, for example, in Homsi et al., Clinical Cancer Research 2007, 13(19): 5855-5861 and Numbenjapon et al., 2009 Clinical Cancer Research 15(13): 4365-4373, herein incorporated by reference in their entireties. Antibiotics including doxycycline, ciprofloxacin, and gentamicin, currently indicated for post-exposure prophylaxis against tularemia or for treatment of pneumonic F. tularensis, can also be used in an analogous manner as meropenem.

To minimize material heterogeneity (i.e., polymer polydispersity and compositional drift), controlled reversible addition-fragmentation chain transfer (RAFT) polymerization is used to prepare the polymer backbones. The RAFT process affords unprecedented control over polymer molecular weight (and polydispersities) and provides polymer chain ends that are telechelic with different end functionalities. The final copolymer compositions can be determined via ¹H NMR spectroscopy. Size exclusion chromatography (SEC) can be used to determine the absolute polymer molecular weights and polydispersity indices (PDI).

The synthetic versatility of the RAFT process allows the preparation of sophisticated polymer architectures with precisely defined molecular weights, discrete block sizes, and copolymer composition. These parameters are systematically varied to evaluate the effect they have on nanoparticle size, stability, drug encapsulation efficiency, and pH-responsive drug release, and intracellular membrane disruption.

Particle assemblies are formulated over a range of concentrations with drug loading densities between 25 and 50 mg/mL. For nanoparticles based on polymer-augmented liposomes, liposomes formulated from phosphatidylcholine and cholesterol in a molar ratio of 1:1 are used. Particle assemblies are aerosolized with the Biaera automated bioaerosol exposure system, and are used for rodent challenges. Aerosol parameters can include the mass mean aerodynamic diameter (MMAD), geometric standard deviations (GSD) and peak particle counts (ppe). Aerosol samples are collected on glass filters to determine drug content spectrophotometrically. Aerosol size and nanoparticle stability are evaluated as a function of nebulizer airflow.

Particle assemblies are evaluated for aqueous stability, biocompatibility, and pH dependent membrane destabilizing activity. For example, the optimum particle-stabilizing random copolymer to core random copolymer ratio and core random polymer compositions (e.g., DEAEMA to BMA copolymer ratio) are evaluated for particle size and pH-dependent red blood cell lysis. For the polymer-augmented liposomes, the optimum polymer loading relative to lipids is determined as a function of pH and block copolymer composition/architecture.

Dynamic light scattering measurements determines nanoparticle size and distribution as well as to follow particle stability as a function of time and storage environment. Static light scattering measurements determines nanoparticle molecular weight (Mw). Nanoparticle morphology is visualized via Transmission Electron Microscopy (TEM).

To establish the modular nature of the polymer-augmented liposome therapeutic nanoparticle systems, antibiotic encapsulation efficiencies are determined for a range of additional antibiotic agents including ciprofloxacin, doxycycline, ceftazidime, meropenem, streptomycin, trimethoprim, and sulfamethoxazole. The introduction of PEGMA (950 Da) is evaluated as PEG has been shown to increased stability of liposomes in lung tissue. Endosomal and lysosomal esterases are added to random copolymers with conjugated therapeutic agents to establish polymeric prodrug cleavage rates. The effect of key formulation parameters on the rate of drug release is evaluated as a function of pH.

The pH-responsive membrane destabilizing activity of the random copolymers is assayed using a red blood cell hemolysis assay. Three different pH conditions are used to mimic endosomal pH environments: extracellular pH) 7.4, early endosome pH) 6.6, late endosome pH) 5.8. The dependence of hemolytic properties on the morphology of the polymer constructs in their self-assembled state is evaluated to optimize the hydrophilic-hydrophobic balance as well as the effect of polymer morphology.

### Example 7

### In Vitro Studies of Antibiotic-Loaded Particle Assemblies

To characterize the fate of internalized random copolymers in the alveolar macrophage, the glycan-targeted particle assemblies of Example 6 are dual-labeled with a pH-sensitive and a pH-insensitive fluorophore and incubated with MH-S cells, an SV40-transformed BALB/c alveolar macrophage cell line. Live-cell fluorescence microscopy using ratiometric imaging obtains a histogram of pH micro environments where the particle assemblies are found versus time to determine the extent of cytosolic delivery. Subcellular fractionation studies directly correlate the intracellular trafficking dynamics of tritium-labeled particle assemblies and the therapeutic agent by measuring radioactivity in isolated vesicular compartments versus the cytosolic compartment. In vitro toxicity is evaluated in MH-S cells.

### Determination of In Vitro Dose Response

Cells are treated with increasing concentrations of loaded particle assemblies for 24 hours. Intracellular concentrations of antibiotics are measured, and doses that achieve the known MIC of the free antibiotic are determined to define the lowest dose used in subsequent experiments. An assumption is made that the intracellular concentration of any antibacterial agent has to be in the range of the conventional minimal inhibitory concentration (MIC), recognizing that pH, protein binding, and other factors may influence efficacy.

### Toxicity Studies

MH-S cells will be incubated with increasing concentrations of loaded particle assemblies for 24 hours. Three concentrations will be used over a 2 log range, where the starting dose is the lowest dose that achieves intracellular MIC as determined above. Controls include PBS, free antibiotic, and carrier alone. Assays are performed in triplicates and repeated at least 3 times. All results are confirmed using primary mouse alveolar macrophages. Cell viability (MTT assay), apoptosis, cell supernatant LDH are measured using standard assays, and cell morphology is assessed. The highest dose that does not cause cell toxicity is determined. Loaded particle assembly morphologies/compositions showing maximum intracellular antibiotic levels are selected for further studies.

### Macrophage Phagocytosis Assay

The ability of alveolar macrophage to phagocytose apoptotic neutrophils is important to resolution of inflammation. Therefore, the ability of macrophages to phagocytose fluorescently labeled beads using standard phagocytosis flow cytometry and fluorescent microscopy assays is studied to determine average number of phagocytosed beads per cell.

### Response to Lipopolysaccharide (LPS)

When faced with larger numbers of infectious particles alveolar macrophages synthesize and secrete a wide array of cytokines including TNF-α, IL-β, and IL-6. To determine the effect of TDS on the production of cytokines from LPS-stimulated cells, MH-S cells are plated onto 24-well plates (1×106 cells/well), pretreated in the presence or absence of TDS for 24 h, and then stimulated with LPS (1 μg/mL) for 4, 12 and 24 h. At each time point, cell-free supernatants are collected and the concentrations of cytokines TNF-α, IL-β, and IL-6 will be measured by ELISA.

### Example 8

### In Vivo Studies of Antibiotic-Loaded Particle Assemblies

### Loaded Particle Assembly In Vivo Toxicology

Delivery of drugs via nebulization occurs by the generation of droplet mists through the use of compressed air or oxygen. This technique consists of dispersing solid or phase-separated drug delivery systems into droplets suspended in a small amount of medium. The subject inhales the mist through the nose and mouth. This route can deliver large quantities of solutions and suspensions as small droplets and requires minimal patient coordination.

BALB/c mice are exposed to three concentrations of aerosolized loaded particle assemblies of Example 6 over a two log range (doses informed by in vitro studies described in Example 7) or controls (PBS, free antibiotic or carrier alone) via an InTox (Moriarty, NM) nose-only exposure chamber. Although this system is more labor intensive than whole animal exposure chamber, nose-only chambers requires less drug per animal. Loaded particle assemblies are nebulized using clinical miniHeart Hi-Flo nebulizers that reliably generate particles of respirable size (2-3 μm). Airflow will be maintained at 8 L/min at 40 psi, and total airflow will be maintained at 19.5 L/min by blowing in 11.5 L/min of humidified dilution air until nebulization is complete. Humidity in dilution air is controlled by passage through a heated humidification chamber. All pressures and flows are controlled and recorded by a Biaera Technologies AeroMP computer-regulated interface.

Aerosol samples collected in impinger tubes are used to estimate aerosol concentrations and drug dose. Since there is subject-to-subject variability in the distribution of reagents delivered, groups of 5 mice per time-point are estimated to be the minimal number that yields reliable results for this study.

### Lung and Systemic Antibiotic Concentrations and Half-Life

Levels of the delivered therapeutic agent are measured in lung homogenates, bronchoalveolar lavage (BAL) fluid, and in isolated alveolar macrophages by the Pharmacokinetics Laboratory at 1 and 24 hours after drug administration. Doses that achieve appropriate MIC in alveolar macrophages are used for toxicity studies listed below. Since the alveolar macrophage has a lifespan of >90 days, there is the potential of establishing a long-lived drug depot. Therefore, to determine the half-life of the intracellular antibiotic, loaded particle assemblies are administered and mice are followed from 48 h to 14 days post-administration.

### Systemic Toxicity

To determine systemic toxicity, mice are weighed and inspected daily, and ventral surface temperature is measured with an infrared thermometer. Each animal is scored for appearance (grooming, piloerection, ocular or nasal discharge, respiratory distress, posture), mobility, social activity, and resistance to handling). Animals that lose more than 10 percent of body weight are euthanized.

### Inflammatory Markers and Histopathology

Liver function tests (GOT, GPT, ALT and AST), Cr, peripheral WBC count and differential from cardiac puncture are measured. Lungs from exposed mice are inflated, fixed, paraffin embedded, sectioned and stained with hematoxylin and eosin (H&E), for evaluation of basic lung morphology, and inflammation, using a previously developed scoring system. Bronchoalveolar lavage is performed for measurement of total cell count and cell differential.

Assessment of lung permeability include BAL total protein, IgM, and FITC dextran extravasation after intravenous (IV) injection. BAL cytokine levels re measured using the Fluorokine MultiAnalyte Profiling (MAP) Multiplex Mouse Cytokine Panel. In whole lung homogenates, inflammatory markers including chemokines and pro-inflammatory cytokines are assessed by ELISA or Q-PCR. The highest administered dose that does not cause local or systemic toxicity is determined.

### Biodistribution and Drug Pharmacokinetics (PK)

Near IR probes suitable for conducting noninvasive live animal imaging are integrated into the polymeric scaffolds via reaction of commercially available maleimide fluorophore with 1-2 mol percent pyridyl disulfide functional monomers. Radiolabeled constructs are prepared by quaternization of tertiary amine functionality (located in the pH-responsive segment) with commercially available 3H iodoacetamide. Radiolabeled therapeutic agents are purchased from American Radiolabeled Chemicals. Animals are bled from the tail vein at 0.1, 1, 4, 24, and 48 hours after aerosolized delivery and groups of 5 are euthanized. Organs (liver, heart, lungs, spleen, kidneys, stomach, small and large intestines, bladder, quadriceps muscle) are excised, weighed, homogenized and total fluorescence or radioactive counts are measured and expressed as percentage of starting material. PK parameters are calculated using compartmental and non-compartmental models using the WinNonLin statistical program designed for this purpose (Pharsight Software), a strength of this software is the ability to estimate the effects of different doses and dosing schedules on PK parameters, which assist design of optimal treatment regimens. Parameters to be measured include the plasma half-life (t1/2 a, ˜, y), maximum concentration in plasma (Cmax), area under the plasma concentration curve (AUC), volume of distribution (VD), and plasma clearance (CL) of these reagents. Therapeutic agent levels in BAL fluid, alveolar macrophages, lung homogenate and serum are determined.

### Example 9

### Evaluation of Antibiotic-Loaded Particle Assemblies Targeting

Uptake experiments are conducted with polymeric mannose and galactose functionalized particle assembly derivatives to confirm selective engagement of mannose-specific macrophage surface receptors. Flow cytometry is used to identify specific chemical, architectural, and morphology particle assembly parameters that lead to the highest uptake levels in alveolar macrophages.

Fluorescence microscopy experiments are used to provide direct visualization of targeted and untargeted particle assemblies. These studies are conducted in parallel with intracellular trafficking studies, and confirm the ability of the polymeric mannose derivatives to specifically access intracellular compartments.

### Example 10

### Evaluation of In Vitro and Ex Vivo Efficacy of Antibiotic-Loaded Particle Assemblies in Infected Cell Models

To facilitate study of the highly virulent Tier 1 select agents Francisella tularensis and Burkholderia pseudomallei, surrogate models of infection with closely related pathogens that can be studied under BSL-2 conditions have been developed. F. tularensis subspecies novicida is attenuated in humans in comparison with fully virulent F. tularensis (such as the SchuS4 strain that is common in North America), but is equally virulent in mice. Airborne infection of mice with either strain results in exponential bacterial replication in the lungs, liver, and spleen, and death within 5 days. Similarly, Burkholderia thailandensis is a rare human pathogen, but virulent in mice at high doses and serves as a surrogate for B. pseudomallei. These surrogate models have been used to study host immunity and bacterial virulence.

### In Vitro and In Vivo Models Evaluating Efficacy of Loaded Particle Assemblies

Both F. tularensis and B. pseudomallei infect and replicate in alveolar macrophages. Furthermore, both organisms induce macrophage death as a means to avoid killing. Thus, the efficacy of therapeutic agent-loaded particle assemblies of Example 6 is studied in infected alveolar macrophages in vitro by examining both intracellular bacterial survival/replication and cell death. To examine whether treatment with the loaded particle assemblies increases the killing efficiency of macrophages, MH-S cells are treated with loaded particle assemblies 1 hour before (pre-treatment) or 1 hour after (post-treatment) F. tularensis subspecies novicida U112 or B. thailandensis E264 infection. Bacteria are added to untreated, pre-treated, and post-treated cells at multiplicities of infection of 1:1, 10:1, and 100:1 and incubated at 37° C. for 1 hour. Extracellular bacteria are then removed by serial washing of cells. In some experiments, gentamicin (Francisella) or meropenem (Burkholderia) are added to the media to kill residual extracellular bacteria and to provide comparison conditions. Macrophages treated with loaded particle assembly but not infected are used as a control. At 2, 6, and 12 hours after infection, cells are washed and lysed with 0.5 percent Triton X-IOO; intracellular survival are determined by plating serially diluted cultures, and colonies counted after 72 hours of incubation on trypticase soy agar (TSA) supplemented with L-cysteine (Franciscella) or lysogeny broth (LB) (Burkholderia). These experiments will be repeated using a range of doses of TDS, selected based on earlier experiments. The results are confirmed using freshly isolated murine alveolar macrophages.

To further test the efficacy of loaded particle assemblies against Francisella and Burkholderia infection, loaded particle assemblies are administered to mice by aerosol and subsequently harvest alveolar macrophages at serial time points (1 to 7 days) following treatment. The macrophages are cultured ex vivo and infected with Francisella or Burkholderia as described above.

Identical assays of bacterial survival/replication, and cell toxicity are performed. As above, failure to achieve a reduction in bacterial counts or detection of increased cell toxicity results in revisiting the optimal concentration of loaded particle assemblies.

In response to in vitro and ex vivo experiments, loaded particle assembly composition, drug loading, and morphology are further refined to yield the maximum bacteria killing potency while maintaining nanoparticle stability and biocompatibility.

### Example 11

### Aerosol Challenge Studies of Antibiotic-Loaded Particle Assemblies

The viability and consistency of bacterial suspensions after aerosolization are tested in a Biaera automated whole animal bioaerosol exposure system. Bacterial suspensions in PBS are tested at 3 concentrations over a 2-log range. Aerosol samples are collected in a glass impinger tube and quantitatively cultured. The ratio of the concentration of bacteria in the impinger tubes to the concentration in the nebulizer are used to calculate the spray factor and aerosol exposure concentration.

A whole animal exposure chamber is used for challenge studies as it mimics field exposure to an airborne biothreat. At least 32 mice can be exposed simultaneously in the Biaera chamber. BALB/c mice are used because they are highly susceptible to pulmonary infection with Francisella and Burkholderia species. Studies are performed to estimate bacterial deposition in the lungs after exposure of groups of 5 mice to infectious aerosols generated from a range of starting concentrations of bacteria. Immediately after exposure, the mice are euthanized with an overdose of pentobarbital, exsanguinated by cardiac puncture, and the left lung are harvested and homogenized in 1 ml PBS for quantitative culture. Aerosol samples collected in impinger tubes are cultured in each experiment to estimate aerosol concentrations and exposure dose. Thus, inhalation challenge dosing are measured by two means: calculation of the aerosol exposure concentration and direct measurement of bacterial deposition in lung tissue of sentinel animals.

### Determination of Median Lethal Dose (MLD)

Prior studies with F. novicida have established that deposition of fewer than 10 CFU/lung is uniformly lethal within 5 days. The MLD is assumed to be 10 CFU and 10 MLDs will be assumed to be 100 CFU/lung. The MLD of B. thailandensis is determined by exposing mice to a log₂ range of doses of aerosolized bacteria that bracket the MLD, estimated to fall between 10⁴ and 3×10⁵ CFU/lung. The MLD is calculated from the accumulated data using the method of Reed and Muench.

The efficacy of pre- and post-exposure prophylaxis with therapeutic agent-loaded particle assemblies is tested in two phases. First, loaded particle assemblies found to be effective in vitro is tested in vivo according to the schedule outlined in Table 2.

TABLE 2
In Vivo Challenge Schedule

| Exp. | Loaded Particle | Bacterial | Duration of |
|---|---|---|---|
| # | Assembly Dose | Infection | Observation |
| 1 | Day −3 | Day 0, hour 0 | 14 days |
| 2 | Day −1 | Day 0, hour 0 | 14 days |
| 3 | Day 0, hour −1 | Day 0, hour 0 | 14 days |
| 4 | Day 0, hour +1 | Day 0, hour 0 | 14 days |
| 5 | Day 0, hour +4 | Day 0, hour 0 | 14 days |
| 6 | Day +1 | Day 0, hour 0 | 14 days |

Each experiment compares two groups of 12 mice: those treated with a single dose of aerosolized loaded particle assemblies, and control mice exposed to aerosolized carrier buffer (PBS). Mice are exposed to aerosolized loaded particle assemblies or PBS in a nose-only exposure chamber, then exposed to 10 MLDs of aerosolized bacteria in the whole animal exposure chamber, according to the schedule in Table 2. Mice are observed daily for morbidity and survival. After 14 days, mice are euthanized for harvest of lung, liver, and spleen, which are homogenized for quantitative culture. This strategy is repeated for each loaded particle assembly formulation and dosage to be tested and for each bacterial pathogen (F. novicida and B. thailandensis).

Additional loaded particle assembly dosing points are tested based as warranted (e.g., Day −7, Day +2). Survival in treatment and control groups are compared by log-rank test.

Treatment protocols that demonstrate efficacy (enhanced survival) in screening experiments are further tested in comparison with additional control groups: particle assembly without antibiotic, free antibiotic(s) in PBS, and PBS. Two experimental designs will be used:

Each experiment will compare four groups of 8 mice each: loaded particle assembly, particle assembly without antibiotic, free antibiotic in PBS, and PBS; plus 2 mice to measure bacterial deposition.

Mice are exposed to aerosolized loaded particle assembly or controls in the nose-only exposure system, then challenged with 10 MLDs of aerosolized bacteria in the whole animal chamber. Mice are monitored for morbidity and mortality. After 14 days surviving animals are euthanized. Lungs, liver, and spleen will be harvested for quantitative culture and histopathology.

The effect of loaded particle assembly prophylaxis on bacterial containment and the host response to infection are determined from timed tissue harvests. Each experiment compares four groups of 8 mice each: loaded particle assembly, particle assembly without antibiotic, free antibiotic in PBS, and PBS; plus 2 mice to measure bacterial deposition. Mice are exposed to aerosolized loaded particle assembly or controls in the nose-only exposure system and challenged with 10 MLDs of aerosolized bacteria in the whole animal chamber. Four mice from each group are euthanized 3 and 7 days after infection. The left lung and portions of liver and spleen are weighed and homogenized for quantitative culture. The right lung and the remaining portions of liver and spleen are fixed in paraformaldehyde, embedded in paraffin, and sectioned for histological analysis. Serum is tested for evidence of hepatic and renal injury, and tissue samples are tested for drug levels. Bacterial burdens and other measures are compared among groups by ANOVA using GraphPad software.

While illustrative embodiments have been illustrated and described, it will be appreciated that various changes can be made therein without departing from the spirit and scope of the invention.

1-20. (canceled)

21. A random copolymer, comprising
(i) at least one constitutional unit selected from cationic constitutional units, anionic constitutional units, any combination thereof, and hydrophobic uncharged constitutional units, and
(ii) constitutional units comprising a covalently-bound antibiotic agent or kinase inhibitor,
wherein the antibiotic agent or kinase inhibitor is cleavable from the random copolymer by an enzyme or water.

22. The random copolymer of claim 21, wherein the random copolymer comprises cationic constitutional units, hydrophobic uncharged constitutional units, and constitutional units comprising a covalently-bound antibiotic agent or kinase inhibitor.

23. The random copolymer of claim 21, comprising greater than 0 to 90 mole percent of cationic constitutional units, greater than 0 to 70 mole percent of hydrophobic uncharged constitutional units, and greater than 0 to 50 mole percent of constitutional units comprising a covalently-bound antibiotic agent or kinase inhibitor, wherein a total mole percentage of the cationic constitutional units, hydrophobic uncharged constitutional units, and constitutional units comprising a covalently-bound antibiotic agent or kinase inhibitor is 100 mole percent.

24. The random copolymer of claim 21, wherein the random copolymer has a positive or negative net charge.

25. The random copolymer of claim 21, wherein the random copolymer has a neutral net charge.

26. A random copolymer, comprising:
(i) hydrophobic uncharged constitutional units, cationic constitutional units, or anionic constitutional units, or any combination thereof,
(ii) hydrophilic constitutional units, and
(iii) constitutional units comprising a covalently-bound antibiotic agent or kinase inhibitor;
wherein the antibiotic agent or kinase inhibitor is cleavable from the random copolymer by an enzyme or water.

27. The random copolymer of claim 26, comprising greater than 0 to 90 mole percent of hydrophobic uncharged constitutional units, greater than 0 to 90 mole percent of hydrophilic constitutional units, and greater than 0 to 50 mole percent of constitutional units comprising a covalently-bound antibiotic agent or kinase inhibitor, wherein a total mole percentage of hydrophobic uncharged constitutional units, hydrophilic constitutional units, and constitutional units comprising a covalently-bound antibiotic agent or kinase inhibitor is 100 mole percent.

28. The random copolymer of claim 26, comprising greater than 0 to 45 mole percent of anionic constitutional units, greater than 0 to 45 mole percent of cationic constitutional units, greater than 0 to 50 mole percent of hydrophilic constitutional units, and greater than 0 to 50 mole percent of constitutional units comprising a covalently bound antibiotic agent or kinase inhibitor, wherein a total mole percentage of anionic constitutional units, cationic constitutional units, hydrophilic constitutional units, and constitutional units comprising a covalently-bound antibiotic agent or kinase inhibitor is 100 mole percent.

29. The random copolymer of claim 26, comprising greater than 0 to 70 mole percent of anionic constitutional units, greater than 0 to 70 mole percent of hydrophilic constitutional units, and greater than 0 to 50 mole percent of constitutional units comprising a covalently bound antibiotic agent or kinase inhibitor, wherein a total mole percentage of anionic constitutional units, hydrophilic constitutional units, and constitutional units comprising a covalently-bound antibiotic agent or kinase inhibitor is 100 mole percent.

30. The random copolymer of claim 26, comprising greater than 0 to 70 mole percent of cationic constitutional units, and greater than 0 to 70 mole percent of hydrophilic constitutional units, and 0 to 50 mole percent of constitutional units comprising a covalently bound antibiotic agent or kinase inhibitor, wherein a total mole percentage of cationic constitutional units, hydrophilic constitutional units, and constitutional units comprising a covalently-bound antibiotic agent or kinase inhibitor is 100 mole percent.

31. The random copolymer of claim 26, wherein the anionic constitutional unit is selected from 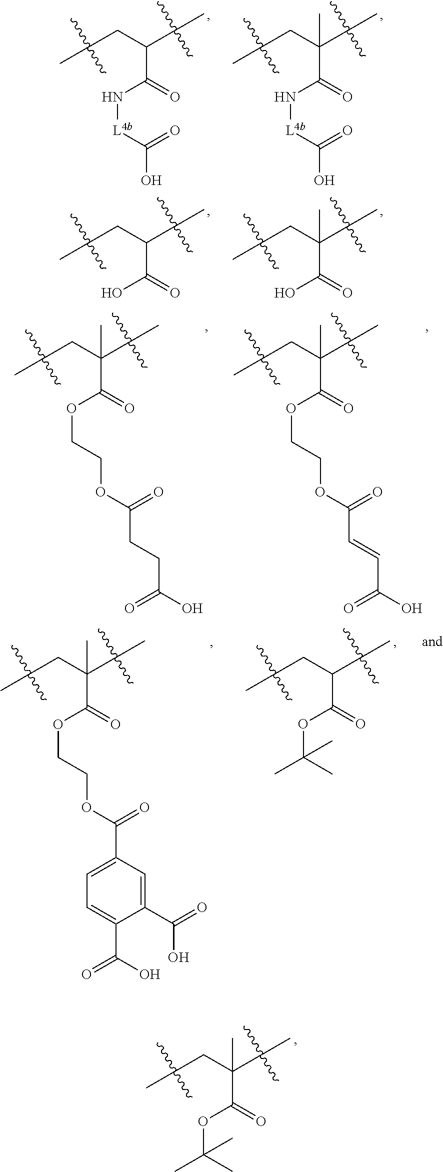
wherein L^{4b} is selected from absent, C₁₋₆ alkylene, C₂₋₆ alkenylene, and arylene, and wherein said C₂₋₆ alkenylene or arylene is optionally substituted with 1, 2, 3, or 4 substituents independently selected from halo and COOH.

32. The random copolymer of claim 26, wherein the cationic constitutional unit is selected from the group consisting of 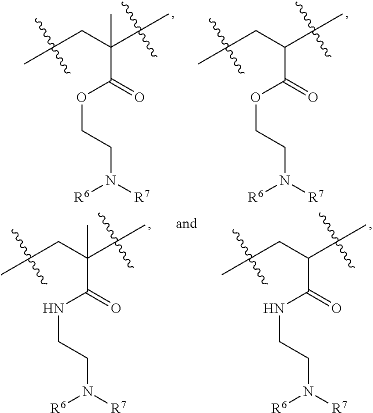
wherein R⁶ and R⁷ are each independently selected from H and C₁₋₆ alkyl.

33. The random copolymer of claim 26, wherein the hydrophobic uncharged constitutional units each comprises a C₈-C₂₆ fatty acid side chain.

34. The random copolymer of claim 26, wherein the hydrophilic constitutional units comprise poly(alkylene glycol) having at least 5 alkylene glycol constitutional units.

35. (canceled)

36. The random copolymer of claim 26, wherein the hydrophilic constitutional units comprise a polysaccharide.

37. The random copolymer of claim 26, wherein the antibiotic agent is selected from the group consisting of ciproflaxin, meropenem, doxycycline, and ceftazidime.

38. The random copolymer of claim 26, wherein the kinase inhibitor is dasatinib.

39. The random copolymer of claim 26, wherein the antibiotic agent or kinase inhibitor is conjugated to the copolymer via an ester linkage, hydrazone linkage, or an amide linkage.

40. The random copolymer of claim 26, optionally comprising a targeting agent selected from the group consisting of transferrin, glycan, biotin, folic acid, and vitamin B.

41. The random copolymer of claim 26, wherein when the random copolymer releases the antibiotic agent or the kinase inhibitor in a linear manner over a period of 1 to 900 hours, when subjected to physiological conditions.

42-46. (canceled)
